(19) United States
(12) Patent Application Publication
(10) Pub. No.: US 2010/0004728 A1
(43) Pub. Date: Jan. 7, 2010

(54) GRAFT ENDOFRAME HAVING AXIALLY VARIABLE CHARACTERISTICS

(75) Inventors: K.T. Venkateswara Rao, San Jose, CA (US); Anant Kumar, San Jose, CA (US); Ivan Tzvetanov, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Nellix, Inc., Palo Alto, CA (US)

(21) Appl. No.: 12/371,087

(22) Filed: Feb. 13, 2009

Related U.S. Application Data
(60) Provisional application No. 61/028,453, filed on Feb. 13, 2008.
(60) Provisional application No. 61/029,225, filed on Feb. 15, 2008.

(51) Int. Cl. A61F 2/06; B44C 1/22

(52) U.S. Cl. 623/1.11; 623/1.16; 623/1.3; 216/94

(57) ABSTRACT

A prosthesis comprises a tubular body that is expandable from a contracted configuration to a radially expanded configuration. The tubular body has a total length and comprises a first section, a second section and a central section disposed therebetween. The total length of the tubular body in the expanded configuration is at least 95% of the total length of the tubular body in the contracted configuration. The three sections have a plurality of tubular rings, each with a plurality of struts having a length and coupled together to form a series of peaks and valleys. A connector couples adjacent tubular rings together. The length of the central section struts is different than the length of the other struts and the central section is coupled with both the first and second sections.

### CROSS-REFERENCES TO RELATED APPLICATIONS

The present application is a non-provisional of and claims the benefit of U.S. Provisional Application No. 61/028,453 filed Feb. 13, 2008 (Attorney Docket No. 025925-002700US). The present application is also a non-provisional of, and claims the benefit of U.S. Provisional Application No. 61/029,225 filed Feb. 15, 2008 (Attorney Docket No. 025925-002710US). The entire contents of each of the provisional patent applications listed above is incorporated herein by reference.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK.

NOT APPLICABLE

### BACKGROUND OF THE INVENTION

1. Field of the Invention

The present invention relates generally to medical apparatus and methods for treatment. More particularly, the present invention relates to prostheses and methods for treating aneurysms.

Aneurysms are enlargements or “bulges” in blood vessels which are often prone to rupture and which therefore present a serious risk to the patient. Aneurysms may occur in any blood vessel but are of particular concern when they occur in the cerebral vasculature or the patient's aorta.

The present invention is particularly concerned with aneurysms occurring in the aorta, particularly those referred to as aortic aneurysms. Abdominal aortic aneurysms (AAA's) are classified based on their location within the aorta as well as their shape and complexity. Aneurysms which are found below the renal arteries are referred to as infrarenal abdominal aortic aneurysms. Suprarenal abdominal aortic aneurysms occur above the renal arteries, while thoracic aortic aneurysms (TAA's) occur in the ascending, transverse, or descending part of the upper aorta.

Infrarenal aneurysms are the most common, representing about eighty percent (80%) of all aortic aneurysms. Suprarenal aneurysms are less common, representing about 20% of the aortic aneurysms. Thoracic aortic aneurysms are the least common and often the most difficult to treat. Most or all present endovascular systems are also too large (above 12 F) for percutaneous introduction.

The most common form of aneurysm is “fusiform,” where the enlargement extends about the entire aortic circumference. Less commonly, the aneurysms may be characterized by a bulge on one side of the blood vessel attached at a narrow neck. Thoracic aortic aneurysms are often dissecting aneurysms caused by hemorrhagic separation in the aortic wall, usually within the medial layer. The most common treatment for each of these types and forms of aneurysm is open surgical repair. Open surgical repair is quite successful in patients who are otherwise reasonably healthy and free from significant co-morbidities. Such open surgical procedures are problematic, however, since access to the abdominal and thoracic aortas is difficult to obtain and because the aorta must be clamped off, placing significant strain on the patient's heart.

Over the past decade, endoluminal grafts have come into widespread use for the treatment of aortic aneurysm in patients who cannot undergo open surgical procedures. In general, endoluminal repairs access the aneurysm “endoluminally” through either or both iliac arteries in the groin. The grafts, which typically have been fabric or membrane tubes supported and attached by various stent structures, are then implanted, typically requiring several pieces or modules to be assembled in situ. Successful endoluminal procedures have a much shorter recovery period than open surgical procedures.

Present endoluminal aortic aneurysm repairs, however, suffer from a number of limitations. A significant number of endoluminal repair patients experience leakage at the proximal juncture (attachment point closest to the heart) within two years of the initial repair procedure. While such leaks can often be fixed by further endoluminal procedures, the need to have such follow-up treatments significantly increases cost and is certainly undesirable for the patient. A less common but more serious problem has been graft migration. In instances where the graft migrates or slips from its intended position, open surgical repair is required. This is a particular problem since the patients receiving the endoluminal grafts are often those who are not considered good candidates for open surgery. Further shortcomings of the present endoluminal graft systems relate to both deployment and configuration. Current devices often have an annular support frame that is stiff and difficult to deliver as well as unsuitable for treating many geometrically complex aneurysms, particularly infrarenal aneurysms with little space between the renal arteries and the upper end of the aneurysm, referred to as short-neck or no-neck aneurysms. Aneurysms having torturous geometries, are also difficult to treat.

For these reasons, it would be desirable to provide improved methods and systems for the endoluminal and minimally invasive treatment of aortic aneurysms. In particular, it would be desirable to provide systems and methods which can be delivered percutaneously and that can track and be deployed in tortuous vessels. It would also be desirable to provide prostheses with minimal or no endoleaks, which resist migration, which are flexible and relatively easy to deploy, and which can treat many if not all aneurismal configurations, including short-neck and no-neck aneurysms as well as those with highly irregular and asymmetric geometries. It would be further desirable to provide systems and methods which are compatible with current designs for endoluminal stents and grafts, including single lumen stents and grafts, bifurcated stents and grafts, parallel stents and grafts, as well as with double-walled filling structures which are the subject of the commonly owned, copending applications described below. The systems and methods would preferably be deployable with the stents and grafts at the time the stents and grafts are initially placed. Additionally, it would be desirable to provide systems and methods for repairing previously implanted aortic stents and grafts, either endoluminally or percutaneously. At least some of these objectives will be met by the inventions described hereinbelow.

2. Description of the Background Art

U.S. Patent Publication No. 2006/0025853 describes a double-walled filling structure for treating aortic and other aneurysms. Copending, commonly owned U.S. Patent Publication No. 2006/0212112, describes the use of liners and extenders to anchor and seal such double-walled filling structures within the aorta. The full disclosures of both these publications are incorporated herein by reference. PCT Publication No. WO 01/21108 describes expandable implants attached to a central graft for filling aortic aneurysms. See also U.S. Pat. Nos. 5,330,528; 5,534,024; 5,843,160; 6,168,592; 6,190,402; 6,312,462; 6,312,463; U.S. Patent Publications 2002/0045848; 2003/0014075; 2004/0204755; 2005/0004660; and PCT Publication No. WO 02/102282.

### BRIEF SUMMARY OF THE INVENTION

The present invention provides apparatus and methods for the treatment of aneurysms, particularly aortic aneurysms including both abdominal aortic aneurysms (AAA) and thoracic aortic aneurysms (TAA).

In a first aspect of the present invention, a prosthesis comprises a tubular body expandable from a contracted configuration to a radially expanded configuration. The tubular body has a total length and comprises a first section, a second section and a central section disposed therebetween. The total length of the tubular body in the expanded configuration is preferably at least 95% of the total length of the tubular body in the contracted configuration, and even more preferably at least 98%. The first section comprises a plurality of tubular rings with each ring comprising a plurality of struts having a length. The struts of the first section are coupled together to form a circumferential series of peaks and valleys and a connector couples adjacent tubular rings together. The second section comprises a plurality of tubular rings with each ring comprising a plurality of struts having a length. The second section struts are coupled together to form a circumferential series of peaks and valleys and a connector couples adjacent tubular rings together. The central section comprises a plurality of tubular rings with each ring comprising a plurality of struts having a length. The central section struts are coupled together to form a circumferential series of peaks and valleys and a connector couples adjacent tubular rings together. The length of the central section struts is different than the length of the first and second section struts. Additionally, the central section is coupled with the first and second sections.

In some embodiments, the length of the first section struts may be greater than the length of both the second section struts and the length of the central section struts. Also the length of the central section struts may be greater than the length of the second section struts so that the first section may have a diameter in the expanded configuration that is greater than a diameter of the second and central sections in the expanded configuration. The diameter of the central section in the expanded configuration may be greater than the diameter of the second section in the expanded configuration, and the first section may be adapted to radially expand first, followed by radial expansion of the central section which is followed by radial expansion of the second section.

Sometimes the tubular body may comprise a stepped region between an outer surface of the first section in the expanded configuration and an outer surface of the central section in the expanded configuration. The stepped region may also be between an outer surface of the central section in the expanded configuration and an outer surface of the second section in the expanded configuration. In other embodiments, the first section may comprise a first ring and a second ring. The first ring may comprise struts having the first section strut length and the second ring may comprise struts having a length less than the first section strut length. The second ring strut length also may be greater than the second section strut length and the central section strut length so that the tubular body in the expanded configuration may taper substantially uniformly from the first section to the central and second sections.

The central section strut length may be less than both the first section strut length and the second section strut length. Therefore, the central section may be adapted to radially expand after both the first section and the second section radially expand. In other embodiments, the first section strut length and the second section strut length may be greater than the central section strut length. Thus, the first section and the second section may be adapted to radially expand before the central section radially expands.

Sometimes the first section may comprise a first ring and a second ring. The second ring may be closer to the central section than the first ring. The first ring may comprise struts having the first section strut length and the second ring may comprise struts having a length less than the first section strut length. The tubular prosthesis in the expanded configuration may also comprise a first flared end which comprises the first and second rings. The first ring may have an expanded diameter larger than an expanded diameter of the second ring. The second section may comprise a first ring and a second ring with the second ring being closer to the central section than the first ring. The first ring of the second section may comprise struts having the second section strut length and the second ring of the second section may comprise struts having a length less than the second section strut length. Thus, the tubular prosthesis in the expanded configuration may comprise a second flared end opposite the first flared end. The second flared end may comprise the first and second rings of the second section, with the first ring of the second section having an expanded diameter larger than an expanded diameter of the second ring in the second section. Some embodiments may comprise a fourth section. The fourth section may be disposed between the first and central sections or between the central and second sections. The fourth section may comprise a plurality of tubular rings with each ring comprising a plurality of struts having a length. The struts of the fourth section may be coupled together to form a circumferential series of peaks and valleys and a connector may coupled adjacent tubular rings together.

The second section strut length may be less than both the first section strut length and the central section strut length, and the second section may be adapted to radially expand after the first section and the central section radially expand. Some embodiments include a fourth section that may be disposed between the central section and the second section. The fourth section may comprise a plurality of tubular rings with each ring comprising a plurality of struts having a length. The struts of the fourth section may be coupled together to form a circumferential series of peaks and valleys and a connector may couple adjacent tubular rings together. The strut length in the second section and the fourth section may be less than the strut length in the first section and the central section and the first section and the central section may be adapted to radially expand prior to radial expansion of the second and fourth sections.

The central section strut length may be greater than the first section strut length and the second section strut length so that the central section may be adapted to radially expand prior to radial expansion of both the first and second sections.

The tubular body may have a first diameter in the contracted configuration and a second diameter in the expanded configuration. The ratio of the second diameter to the first diameter may be greater than 1 and less than about 15. The tubular body may be balloon expandable. The sections may have a diameter in the radially expanded configuration and each of the sections may be able to maintain at least 50% of their radially expanded diameter when an externally applied differential radial pressure of between about 60 to about 1000 mm of Hg is applied thereto.

In the first section, the peaks of a first tubular ring may be out-of-phase with the peaks in an adjacent tubular ring. The first section may comprise two tubular rings. A first connector may couple the first tubular ring with the second tubular ring and one end of the connector may be coupled with a valley of the second tubular ring. A second connector may couple the second tubular ring with an adjacent tubular ring. One end of the second connector may be coupled with an inside radius of a peak in the second tubular ring. Sometimes the first connector may have first and second ends and the first connector may couple the first tubular ring with the second tubular ring. The first end may be coupled with an inside radius of a peak in the first tubular ring and the second end may be coupled with a valley in the second ring. In still other embodiments, the connector in the first section may have a first end coupled to a valley in a first tubular ring and a second end may be coupled to either a peak or a valley in an adjacent tubular ring. The second end may be coupled to an inside radius of a peak in the adjacent tubular ring. The connector in the first section may comprise a region having a chevron-like shape. The connector may allow the prosthesis to be formed into a curve having a radius of 0.2 inches or more without forming a kink. A kink may comprise a collapsed region of the tubular prosthesis having a diameter in the expanded configuration less than 50% of the diameter of the tubular prosthesis in the expanded configuration. A kink may also comprise a collapsed region of the tubular prosthesis having a cross-sectional area less than 50% of the uncollapsed cross-sectional area.

In the first section, the struts may have a width and the peaks may have a width greater than the strut width. The connector in the first section may have a width and the struts may have a width wider than the connector width. The struts of the first section may have a width and the width may vary along a longitudinal axis of the strut. The struts of the first section may have a first end, a second end opposite thereof and a central region therebetween and strut width may increase from the central region of the strut to either the first end or the second end. The struts of the first section may have a width and the width may be greatest at the peaks.

The central section strut length may be less than the first section strut length. In the central section, the peaks of a first tubular ring may be in-phase with the peaks in an adjacent tubular ring. Sometimes the central section comprises at least four tubular rings. The connector in the central section may have a first end coupled to a peak in a first tubular ring and a second end may be coupled to either a peak or a valley in an adjacent tubular ring. The first end may be coupled to an inside radius of the peak. The connector in the central section may have a first end coupled to a valley in a first tubular ring and a second end may be coupled to either a peak or a valley in an adjacent tubular ring. The connector in the central section may comprise a region having a chevron-like shape. The connector may allow the prosthesis to be formed into a curve having a radius of 0.2 inches or more without forming a kink. The kink may generally take the same form as previously described above.

In the central section the struts may have a width and the peaks may have a width wider than the strut width. Also, in the central section the connector may have a width and the struts may have a width wider than the connector width. The struts of the central section may have a width and the width may vary along a longitudinal axis of the strut. The struts of the central section may have a first end, a second end opposite thereof and a central region therebetween and strut width may increase from the central region of the strut to either the first end or the second end. The struts of the central section may have a width and the width may be greatest at the peaks.

The second section strut length may be less than the central section strut length. In the second section the pitch of the tubular rings may be greater than the pitch of tubular rings in the first or central sections. The peaks of a first tubular ring in the second section may be in-phase with the peaks in an adjacent tubular ring. The second section may comprise four tubular rings. The connector in the second section may have a first end coupled to a peak in a first tubular ring and a second end may be coupled to either a peak or a valley in an adjacent tubular ring. The second end may be coupled to an inside radius of a peak in the adjacent tubular ring. The connector in the second section may have a first end coupled to a valley in a first tubular ring and a second end may be coupled to either a peak or a valley in an adjacent tubular ring. The connector in the second section may comprise a region having a chevron-like shape. The connector may allow the prosthesis to be formed into a curve having a radius of 0.2 inches or more without forming a kink. The kink may generally take the same form as previously described above.

In the second section the struts may have a width and the peaks may have a width wider than the strut width. In the second section the connector may have a width and the struts may have a width wider than the connector width. The struts of the second section may have a width and the width may vary along a longitudinal axis of the strut. The struts of the second section may have a first end, a second end opposite thereof and a central region therebetween and wherein strut width may increase from the central region of the strut to either the first end or the second end. The struts of the second section may have a width and the width may be greatest at the peaks.

The prosthesis may further comprise a cover coupled to at least a portion of the tubular body. The cover may comprise an inflatable member made from a polymer such as ePTFE.

At least one of the connectors in the first, second or central sections may comprise an elongate tapered strut. The connector may also comprise a strut having a chevron-like shape. The widest width of the strut may be at the apex of the chevron. The connector may allow the prosthesis to be formed into a curve having a radius of 0.2 inches or more without forming a kink. The kink may comprise a collapsed region of the tubular prosthesis having a diameter in the expanded configuration less than 50% of the diameter of the tubular prosthesis in the expanded configuration. A kink may also comprise a collapsed region of the tubular prosthesis having a cross-sectional area less than 50% of the uncollapsed cross-sectional area. At least one of the connectors in the first, second or central sections may comprise a strut forming a chevron-like pattern, wherein the strut further comprises a stopping element adapted to prevent the chevron from collapsing. The stopping element may comprise a first raised region of the strut and a second raised region of the strut. The first and second raised regions may be disposed on opposite sides of the chevron.

In another aspect of the present invention, a method for treating an aneurysm in a blood vessel comprises providing a delivery catheter having a prosthesis coupled thereto. The prosthesis comprises a tubular body expandable from a contracted configuration to a radially expanded configuration. The tubular body has a total length and comprises a first section, a second section and a central section disposed therebetween, wherein each of the sections has a longitudinal length. The contracted prosthesis is advanced toward the aneurysm and radially expanding the prosthesis expands each of the first, the central, and the second sections to an expanded diameter. The central section expands to a diameter different than the expanded diameter of the first section and the expanded diameter of the second section. The total length of the tubular body in the radially expanded configuration is preferably at least 95% of the total length of the tubular body in the contracted configuration, and in some embodiments, even more preferably at least 98%. The delivery catheter is then removed from the aneurysm.

The step of radially expanding the prosthesis may comprise radially expanding the first section before radially expanding the central section, and radially expanding the central section before radially expanding the second section. The expanded diameter of the first section may be greater than the expanded diameter of the central section and the expanded diameter of the central section may be greater than the expanded diameter of the second section. The step of radially expanding the prosthesis may comprise forming a stepped region between an outer surface of the first section and an outer surface of the central section. The stepped region may also be between an outer surface of the central section and an outer surface of the second section. Radially expanding the prosthesis may comprise forming a substantially smooth taper from the first section to the central section and the second section.

The step of radially expanding the prosthesis may comprise radially expanding the central section after radially expanding the first section and the second section. Radially expanding the prosthesis may also comprise radially expanding first section and the second section before radially expanding the central section. In some embodiments, the step of radially expanding the prosthesis may comprise flaring at least one of the first section or the second section while in other embodiments, the step of radially expanding the prosthesis comprises radially expanding the second section after radially expanding the first section and the central section. In still other embodiments, the tubular body may further comprise a fourth section that may be disposed between the central section and the second section. The step of radially expanding the prosthesis may comprise radially expanding the first section and the central section prior to radially expanding the second section and the fourth section. In yet another embodiment, the step of radially expanding the prosthesis may comprise radially expanding the central section prior to radially expanding both the first section and the second section.

Radially expanding the prosthesis may comprise expanding the prosthesis from a first diameter in the contracted configuration to a second diameter in the radially expanded configuration such that the ratio of the second diameter to the first diameter may be greater than 1 and less than about 15. Additionally, radially expanding the prosthesis may comprise expanding an expandable member such as a balloon disposed on the delivery catheter.

The tubular prosthesis may have a diameter in the radially expanded configuration and the method may further comprise maintaining at least 50% of the radially expanded diameter along at least a portion of the tubular prosthesis when an externally applied differential radial pressure of between about 60 mm Hg to about 1000 mm Hg is applied thereto. Sometimes, a curve may be formed in the tubular prosthesis. The curve may have a radius of 0.2 inches or more without forming a kink. The kink may comprise a collapsed region of the tubular prosthesis having a diameter in the expanded configuration less than 50% of the diameter of the tubular prosthesis in the expanded configuration. The kink may also comprise a collapsed region of the tubular prosthesis having a cross-sectional area less than 50% of the uncollapsed cross-sectional area.

The prosthesis may further comprise an inflatable member coupled with the tubular body and the method may further comprise inflating the inflatable member. The inflatable member may be filled with an situ curable polymer to a differential pressure of 60-1000 mm Hg, and the expanded prosthesis may still allow blood perfusion therethrough during filling and curing of the inflatable member. The inflatable member may be inflated into engagement with a wall of the aneurysm. Sometimes the inflatable member may be inflated with an in situ curable polymer. The step of inflating may also comprise anchoring the inflatable member and the tubular body with the aneurysm.

The first section of the prosthesis may be disposed upstream of the aneurysm and the central section may be disposed in the aneurysm. The second section may be disposed downstream of the aneurysm. The aneurysm may be disposed in any part of the aorta, including the abdominal aorta. The delivery catheter may comprise a restraining member disposed thereon and radially expanding the prosthesis may comprise removing the restraining member from the tubular prosthesis. Removing the delivery catheter may comprise deflating an inflatable member disposed on the delivery catheter. The prosthesis may comprise a therapeutic agent coupled thereto and the method may further comprise delivering the therapeutic agent in a controlled manner.

In another aspect of the present invention, a method of fabricating a tubular prosthesis having a longitudinal axis and axially variable characteristics comprises fabricating a first region of the tubular prosthesis, the first region having a first set of material characteristics. The method also includes fabricating a second region of the tubular prosthesis, the second region having a second set of material characteristics. Also, the method includes fabricating a third region of the tubular prosthesis, the third region having a third set of material characteristics. The first region, second region and third region are axially aligned along the longitudinal axis, and the first set of material characteristics is different than the second set of material characteristics. The second set of material characteristics is different than the third set of material characteristics. The first region radially expands before the second or the third regions when the tubular prosthesis is radially expanded.

Fabricating the first region, the second region or the third region may comprise electrical discharge machining, laser cutting or photochemical etching of a tube or a substantially flat sheet of material. The second region may be disposed between the first and third regions of the prosthesis.

The method may further comprise fabricating a fourth region of the tubular prosthesis, the fourth region having a fourth set of material characteristics. The fourth set of material characteristics may be different than the first set of material characteristics. Also the fourth region may radially expand after the first region of the tubular prosthesis when deployed. The first, second, third, or fourth set of material characteristics may comprise at least one mechanical property selected from the group consisting of strut length, strut width, strut thickness, number of struts per cell, connector radius, connector thickness, connector geometry, material temper, material strength, and combinations thereof.

These and other embodiments are described in further detail in the following description related to the appended drawing figures.

### BRIEF DESCRIPTION OF THE DRAWINGS

FIG. 1 illustrates an unrolled and flat view of a tubular prosthesis.

FIG. 2 is an enlarged view of the proximal section of the prosthesis in FIG. 1.

FIG. 3 is an enlarged view of the central section of the prosthesis in FIG. 1.

FIG. 4 shows a finite element analysis of stress in an expanded prosthesis.

FIG. 5 is an enlarged view of the connector in the central section of the prosthesis illustrated in FIG. 1.

FIG. 6 is an enlarged view of the distal section of the prosthesis in FIG. 1.

FIG. 7 is an enlarged view of the distal section of the prosthesis shown in FIG. 1.

FIGS. 8-11 illustrate exemplary embodiments of connectors.

FIGS. 12-13 illustrate various connection points for connectors.

FIG. 14 illustrates an alternative embodiment of a tubular prosthesis.

FIGS. 15A-15E illustrate a method of treating an aneurysm.

FIG. 16 illustrates tapered connectors.

FIG. 17 illustrates a stopping element on the connector.

FIG. 18 illustrates the prosthesis of FIG. 14 in the expanded state.

FIG. 19 illustrates expansion of a prosthesis resulting in stepped regions.

FIG. 20 illustrates a smooth, tapered expansion of a prosthesis.

FIGS. 21-26 illustrate other embodiments of a prosthesis having axially variable characteristics.

FIGS. 27A-27C illustrate still another embodiment of a prosthesis having axially variable characteristics.

### DETAILED DESCRIPTION OF THE INVENTION

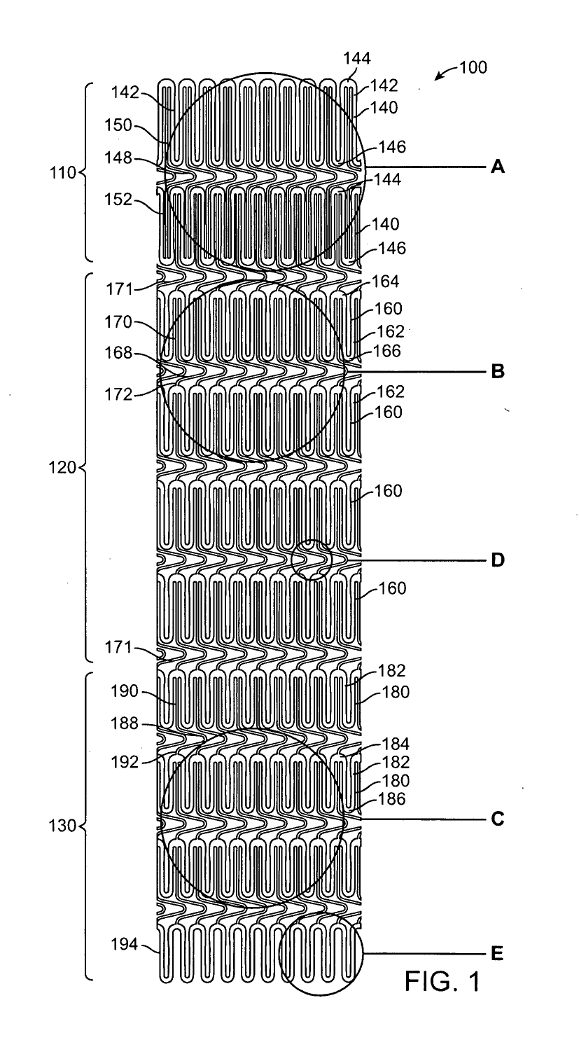

FIG. 1, in accordance with the principles of the present invention, illustrates an exemplary embodiment of a tubular prosthesis 100 having three distinct regions. Prosthesis 100 represents an implantable endoframe that may be used in conjunction with a polymer or fabric cover in the treatment of aneurysms. The prosthesis may be used alone or in combination with another prosthesis placed adjacent thereto. Prosthesis 100 has a neck region 110, a body region 120 and an iliac region 130. Each section is comprised of a number of tubular rings coupled together with a connector and the three regions are also coupled together with a connector. The tubular rings in each region have different open cell geometries in order to vary the mechanical properties of prosthesis 100 axially along its length.

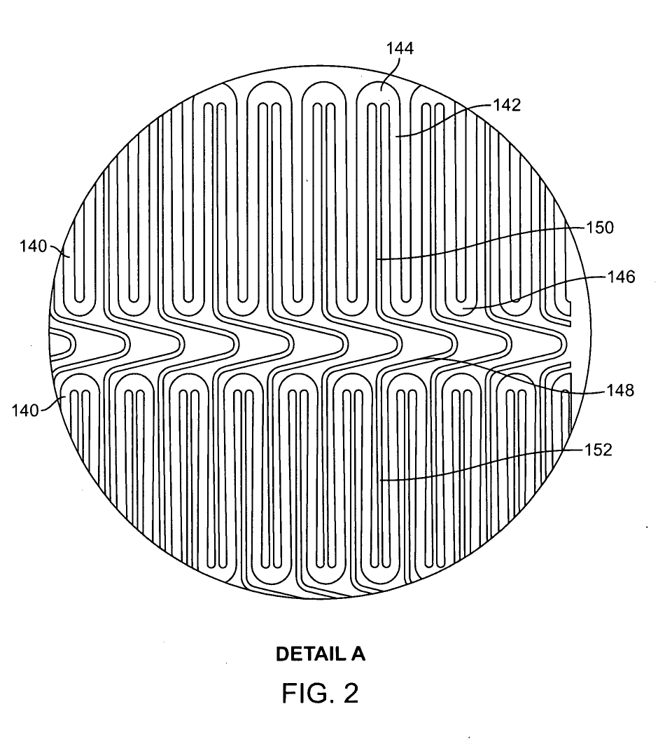

Neck region 110 seen in FIG. 1 and enlarged in FIG. 2, is also referred to as the proximal section because it is often placed proximal to an aneurysm (proximal in this application will refer to the direction closest to the patient's heart). Proximal section 110 comprises two tubular rings 140, although the number of rings may be more or less. Each tubular ring 140 is comprised of a plurality of axially oriented struts 142 coupled together to form a circumferential series of peaks 144 and valleys 146. The struts 142 in the proximal region are longer than the struts 162 in the body region 120 or the struts 182 in the iliac region 130. Having the longest strut length in the proximal section 110 ensures that the proximal section 110 can expand to a larger diameter than the rest of prosthesis 100. Prosthesis 100 may have an expansion ratio up to about 15 to 1. The large expansion ratio in the proximal section 110 allows this section to be further expanded during post-procedure adjustments (e.g. post-procedure dilatation or tacking). Also, having the longest strut length in the proximal section 110 ensures that this portion of the prosthesis radially expands and opens up first during deployment relative to the central section 120 and the distal section 130.

Strut length is optimized so that in the radially expanded configuration, the proximal section 110 can provide adequate radial strength without resulting in excessive stress in the peaks 144 and valleys 146 that would either exceed the ultimate tensile strength of the struts or that compromises the ability of the prosthesis 100 to withstand the cyclic effects of fatigue while implanted in a blood vessel. Typical strut length may range from about 2 mm to about 8 mm long and may range from about 3 mm to about 5 mm long in preferred embodiments.

Keeping the length of strut 142 optimized to provide high radial strength while still permitting the tubular rings 140 to radially expand to the desired diameter results in a large amount of strain at the apex of the peaks 144 and at the bottom of valleys 146. This strain can exceed the material properties of the struts 142 leading to failure. In order to overcome this challenge, the width of the struts 142 at the apex of the peaks 144 and the bottom of valleys 146 may be wider than the rest of strut 142 in order to reduce the strain. Again, strut width must be adjusted carefully because excessive strut width results in a smaller radius of curvature at the strut peak or valley, which in turn leads to undesirable elevated stresses and having more material in the struts 142 also hinders the ability of the tubular rings 140 to be crimped to a lower profile during delivery. Thus, in preferred embodiments the struts 142 are tapered. The strut 142 is thinnest at the circumferential centerline of the tubular ring 140 and tapers outwardly as it extends to either a peak 144 or valley 146. Struts 142 are thickest at the apex of the peaks 144 and at the bottom of valleys 146. Strut width may range from about 0.1 mm to about 1 mm and may range from about 0.2 mm to about 0.5 mm in preferred embodiments. The strut width ratio, defined as the ratio between the widest section and the narrowest section of a strut within one tubular ring may range from about 1.1 to about 4 and may range from about 1.5 to about 2.5 in preferred embodiments.

A connector 148 having axially extending struts 150, 152 couple adjacent tubular rings 140 together. Because the peaks 144 of adjacent tubular rings 140 are out-of-phase with one another, each peak 144 in one tubular ring 140 is coupled with a valley 146 in an adjacent tubular ring 140. The axial struts 150, 152 are substantially parallel to the longitudinal axis of the prosthesis 100. The long length of the axial struts 150, 152 which have a length extending from a peak 144 to a valley 146 in one ring 140 allow adjacent tubular rings greater flexibility than shorter connectors commonly seen in other commercially available prostheses. Connector 148 is shaped like a “v” or a chevron and one axial strut 150 is coupled to an inside radius of a peak 144 while the opposite axial strut 152 is coupled to an inside radius of a valley 146 in the adjacent tubular ring 140. This arrangement of connectors helps to ensure that foreshortening of the proximal section during radial expansion is minimal. In preferred embodiments, foreshortening is about 5% or less and in more preferred embodiments, foreshortening is about 2% or less. The width of connector 148 may range from about 0.025 mm to about 0.3 mm and width may range from about 0.075 mm to about 0.2 mm in preferred embodiments. The ratio of connector width to strut width within a tubular ring having fixed connector widths may range from about 0.1 to about 1.25 and the ratio may range from about 0.2 to about 0.5 in preferred embodiments. In embodiments where the connector width varies due to a taper or other geometry, this ratio may vary from about 0.65 to about 1.0.

Body region 120 in FIG. 1 is also referred to as the central section and comprises four tubular rings 160, although the number of rings may be varied as required. The proximal-most region of the central section 120 is coupled with the distal-most ring in the proximal section 110 via a chevron shaped connector 172. One end of connector 172 is coupled to an outside radius of peak 164 and the other end coupled with an inside radius of peak 144. Each tubular ring 160 is comprised of a plurality of axially oriented struts 162 coupled together to form a circumferential series of peaks 164 and valleys 166. The struts 162 in the central section 120 are shorter than the struts 142 of the proximal section 110, but struts 162 are still longer than the struts 182 in the iliac region 130 of the prosthesis 100, thus the central section 120 begins to expand after the proximal section 110 begins to expand, but before the iliac region 130 expands. Strut length is optimized so that in the radially expanded configuration, the central section can provide adequate radial strength in the central section 120 which is often placed in the sac portion of an aneurysm without resulting in excessive stress in the peaks and valleys that would either exceed the ultimate tensile strength of the struts or that compromises the ability of the prosthesis to withstand the cyclic effects of fatigue while implanted in a blood vessel. The ratio of strut length in the body region to strut length in the proximal region may range from about 0.3 to about 1.0 and may range from about 0.7 to about 0.9 in preferred embodiments.

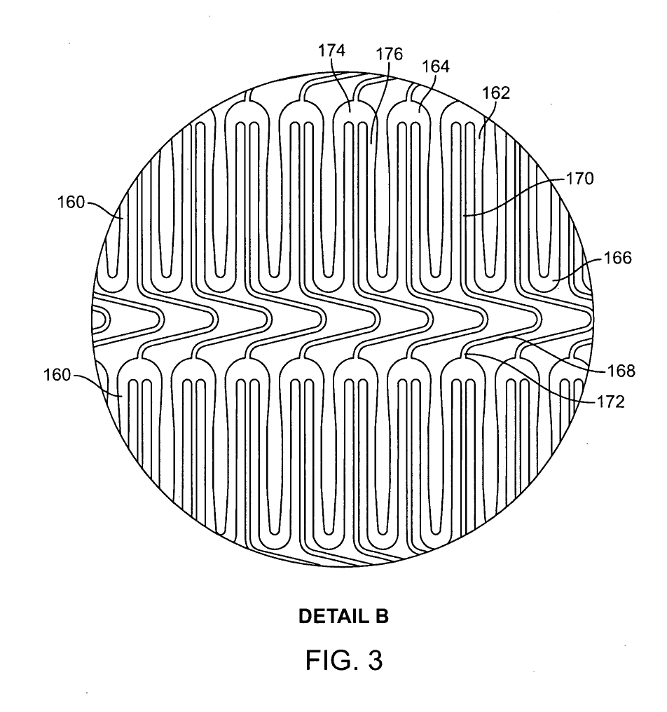

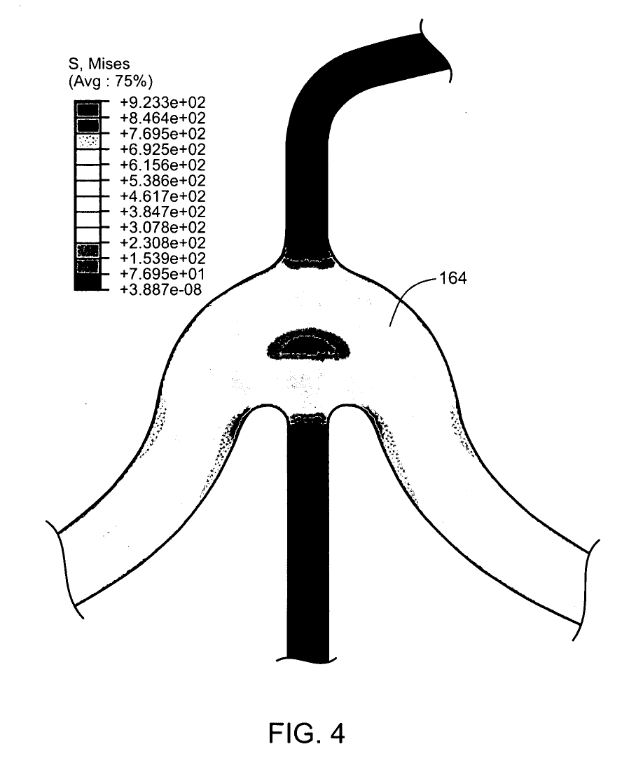

Just as in the proximal section 110, strut length 162 is optimized to provide high radial strength while still permitting the tubular rings 160 to radially expand to the desired diameter without straining the peaks 164 and valleys 166 excessively. Thus, in preferred embodiments the struts 162 are tapered. The strut 162 is thinnest at the circumferential centerline of the tubular ring 160 and tapers outwardly as it extends to either a peak 164 or valley 166. Struts 162 are thickest at the apex of the peaks 164 and at the bottom of valleys 166 and this is illustrated in FIG. 3. Strut width in the body region is similar to that previously described with respect to strut length in the proximal section. FIG. 4 shows the distribution of stress around a peak 164 in an expanded prosthesis 100 as calculated using finite element analysis modeling techniques.

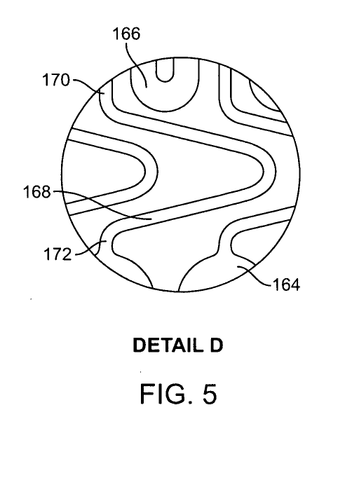

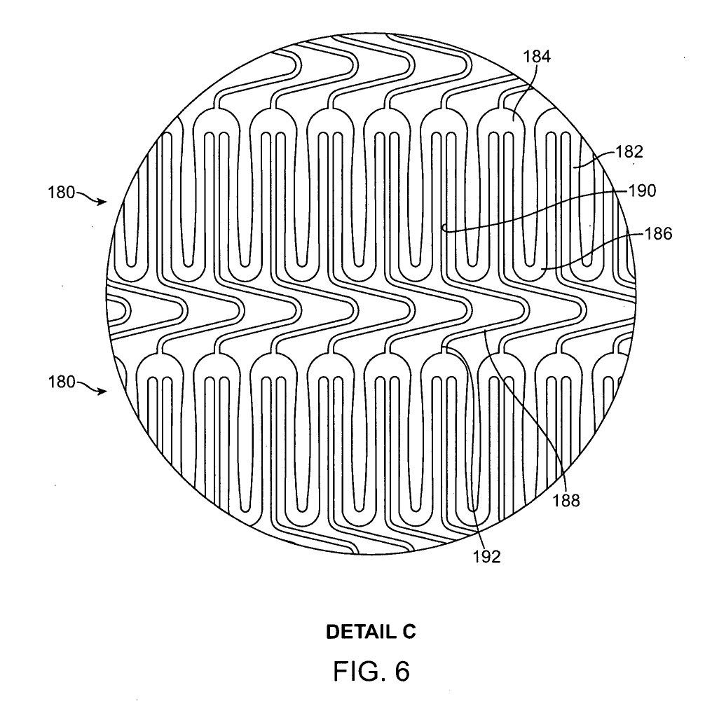

A connector 168 having axially extending struts 170, 172 couple adjacent tubular rings 160 together. In the central section 120, peaks 164 on adjacent tubular rings are in-phase with one another, thus each peak 164 in one tubular ring 160 is coupled with a peak 164 in an adjacent tubular ring 160. However, unlike the proximal section 110, in the central section, one axially extending strut 170 is significantly longer than the other axially extending strut 172 such that the longer strut 170 is coupled to the inside radius of a peak 164 and the shorter strut 172 is coupled to the apex of the outside radius of a peak 164 in an adjacent tubular ring 160. The longer strut 170 is thin enough to nest between adjacent struts 162 on the adjacent tubular ring 160 and this helps reduce profile of the prosthesis when in the crimped configuration. Because the axially extending strut 170 is not a primary load bearing member, it may be considerably thinner than the struts 162. Axially extending struts 170, 172 are also substantially parallel to the longitudinal axis of prosthesis 100 and the connector 168 is shaped like a “v” or a chevron. The arrangement of connectors 168 ensures that there is little or no relative motion between centerlines of adjacent tubular rings 160 and thus foreshortening of the central section 120 is minimal during radial expansion. In this embodiment, foreshortening is about 2% or less. FIG. 5 illustrates the connector 168 in the central section 120 of prosthesis 100. Dimensions of connector 168 are similar to those previously described with respect to the connector 148 in the proximal section.

FIG. 1 also shows the iliac region 130 of prosthesis 100 which may also be referred to as the distal section. The distal section 130 is coupled to the central section 120 via a chevron shaped connector 171. Connector 171 is coupled to the outside radius of peaks 184 and the inside radius of peaks 164. Distal section 130 comprises four tubular rings 180, although this number may be modified as required. Each tubular ring 180 comprises a plurality of axially oriented struts 182 coupled together to form a circumferential series of peaks 184 and valleys 186. The struts 182 in the distal section 130 are the shortest struts in prosthesis 100 as compared with struts 142 in the proximal section and struts 162 in the central section. Because struts 182 are the shortest, the distal section 130 will be the last section of prosthesis 100 to radially expand during deployment. Additionally, shorter struts in the distal section 130 help to ensure more uniform expansion over the circumference of the distal section 130 which is often placed distal to an aneurysm and in or near the iliac arteries where diameter is considerably smaller as compared with the diameter of proximal section 110 which may be placed in the aorta. Using longer struts in the distal section 130 would result in only a few of the struts opening up to match the vessel diameter and this effect can be further exacerbated if the expandable balloon which is used to expand prosthesis 100 is not folded down uniformly. In such instances, the prosthesis 100 will expand in a biased manner to whichever side the balloon fold opens up first and the struts will be wide open on that side while struts on the opposite sides of the prosthesis will remain substantially closed. Using shorter struts as indicated reduces the sensitivity of the distal section 130 to uneven expansion. Additionally, due to the shorter strut 182 length in the distal section 130, the number of rings per linear length, or pitch increases relative to the other sections of the prosthesis 100. This feature has the added benefit of allowing the distal section 130 to accommodate tighter bends in the blood vessel as often seen in the iliac arteries. The ratio of strut length in the distal region to strut length in the body region may range from about 0.3 to about 1.0 and may range from about 0.7 to 0.9 in preferred embodiments.

The struts 182 in the distal section 130 are also tapered like the struts 142 in the proximal section and the struts 162 in the central section. Strut 182 is thinnest at the circumferential centerline of the tubular ring 180 and width tapers outwardly as it extends to either a peak 184 or a valley 186. Struts 182 are therefore thickest at the apex of the peaks 184 and at the bottom of the valleys 186. The widths of struts 182 are similar to those previously described with respect to the struts 162 and 142 in the body and proximal sections of the prosthesis.

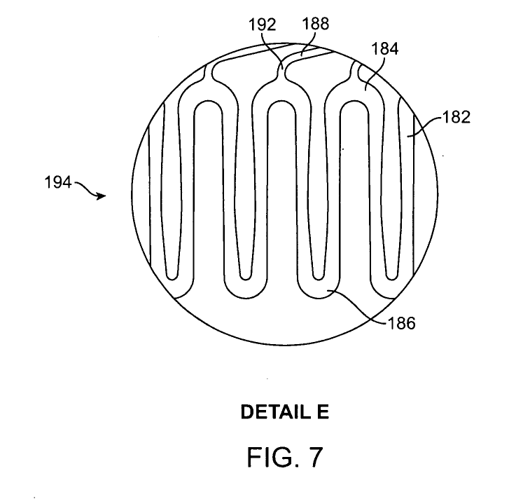

Connector 188 having axially extending struts 190, 192 couples adjacent tubular rings 180 together. In the distal section 130, peaks 184 on adjacent tubular rings are in-phase with one another, thus each peak 184 in one tubular ring 180 is coupled with a peak 180 in an adjacent tubular ring 180. Connector 188 has one axially extending strut 190 which is significantly longer than the other axially extending strut 192, and the longer strut 190 is coupled to the inside radius of peak 184 while the shorter strut 192 is coupled to the outside radius of peak 184 in an adjacent tubular ring 180. Similar to the central section 120, the longer strut 190 is thin enough to nest between adjacent struts 182 in one tubular ring 180 and this helps reduce the profile of the prosthesis 100 in the crimped configuration. Also, the axially extending strut 190 is not a primary load bearing member thus it may be considerably thinner than struts 182. Axially extending struts 190, 192 are also substantially parallel to the longitudinal axis of prosthesis 100 and connector 188 is shaped like a “v” or a chevron. The arrangement of connectors 188 ensures that there is little or no relative motion between centerlines of adjacent tubular rings 180 and thus foreshortening of the distal section 130 is minimal during radial expansion. In this embodiment, foreshortening is about 5% or less, and more preferably 2% or less. Because foreshortening in each of the three sections of the prosthesis limited about 5% or less and more preferably about 2% or less, overall prosthesis length in the expanded configuration will be about 95% or more, and more preferably about 98% or more of the unexpanded prosthesis length. FIG. 5 is an enlarged view of the distal section 130 of prosthesis 100. Additionally, the distal-most tubular ring 194 in the distal section 130 of prosthesis 100 is illustrated in FIG. 7. Because tubular ring 194 is the last ring in distal section 130, it only has connectors 188 coupled to the outside radius of peaks 184. Connector width is similar to that discussed above with respect to connectors in the proximal and body regions of the prosthesis.

The strut thickness in all regions of prosthesis 100 may range from about 0.2 to about 1.0 mm although it may range from about 0.3 mm to about 0.4 mm in preferred embodiments. The aspect ratio between strut thickness to strut width for all regions of prosthesis 100 therefore may range from about 0.3 to about 3 although it may range from about 0.75 at the widest point of the strut to about 2 at the narrowest point of the strut in preferred embodiments.

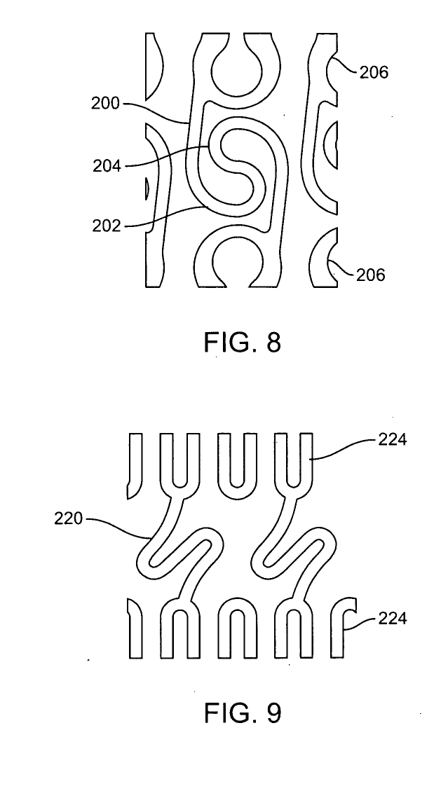

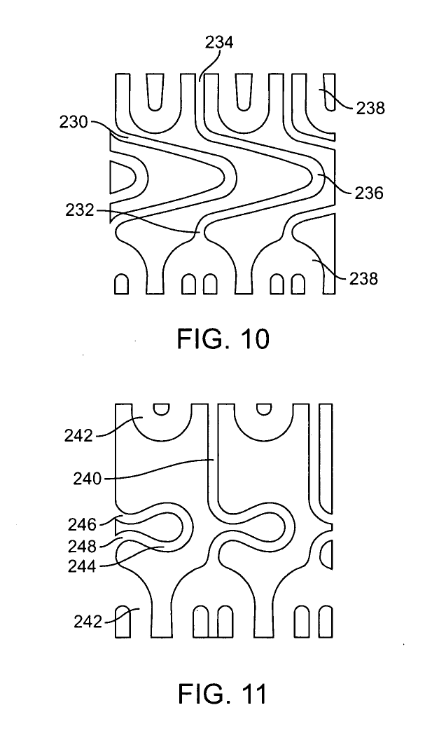

The exemplary embodiment of FIG. 1 describes a “v” shaped or chevron shaped connector which has the advantage of helping the prosthesis to resist kinking. In this or other embodiments utilizing the chevron shaped connector, the prosthesis may be bent into a curve having a radius of 0.2 inches or more without kinking. A kink is defined as a collapsed portion of the prosthesis in the expanded configuration having a diameter less than 50% of the prosthesis diameter in the expanded configuration. A kink may also comprise a collapsed region of the tubular prosthesis having a cross-sectional area less than 50% of the uncollapsed cross-sectional area. Additionally, one of ordinary skill in the art will appreciate that many other connector geometries may also be used. For example, straight connectors or sigmoidal connectors may be used as well as others known to those skilled in the art. FIGS. 8-13 and FIGS. 16-17 illustrate alternative connector embodiments that may be used within the proximal, central or distal sections of the prosthesis or to join the proximal-central sections or central-distal sections together. FIG. 8 shows a connector 200 that may be used to couple adjacent tubular rings 206 together. In this embodiment, the connector 200 has an arcuate shape forming two enlarged head regions 202, 204 that are adjacent one another. The enlarged head regions form a pattern similar to the “yin-yang” symbol and permits the connector 200 to expand axially with minimum peak stress. Similarly, FIG. 9 illustrates another connector embodiment 220 where the arcuate connector 220 forms a sigmoidal shape that also permits axial expansion of the connector 220 between adjacent tubular rings 224. FIG. 10 illustrates yet another embodiment of a connector 230 used to couple adjacent tubular rings 238 together. Connector 230 is similar to the chevron shaped connectors previously described however, in this embodiment, the width of the connector is tapered with thin regions near axially extending struts 232, 234. Strut width increases from the thin regions up to the apex of the chevron 236 where the strut is thickest. The taper design may help to increase axial strength of the connector while still permitting the connector to axially expand and minimizing stress at the apex. FIG. 11 shows a connector embodiment having a nearly closed connector design. In FIG. 11, connector 240 includes an arcuate strut having two legs 246, 248 which form a narrow neck region and an enlarged head region 244. Connector 240 couples adjacent tubular rings 242 together. The enlarged head region 244 allows the connector 240 to axially expand while the narrow neck region formed by legs 246, 248 help prevent the connector 240 from axially collapsing in compression.

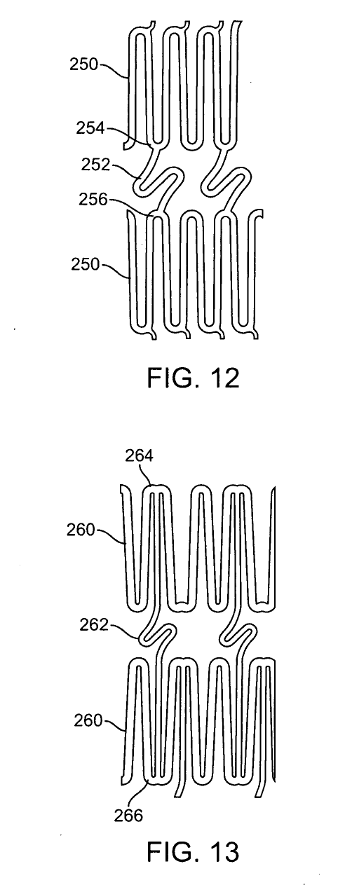

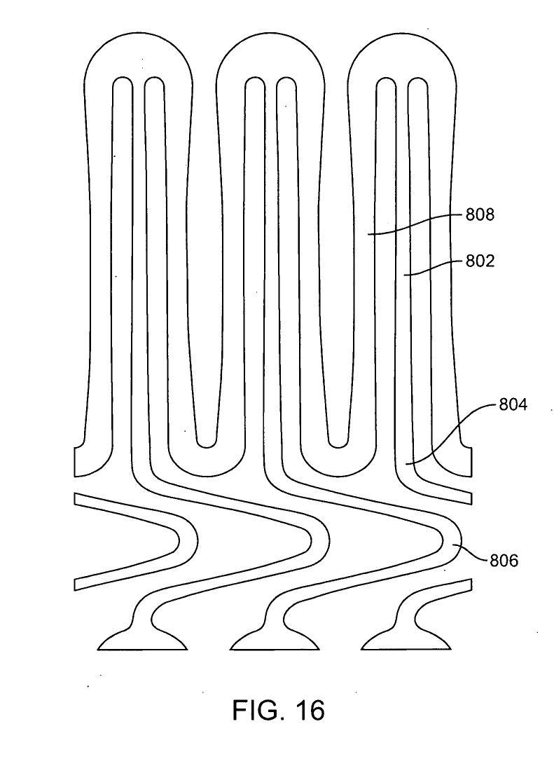

Other connector configurations may also be used to control foreshortening of the prosthesis. For example, FIG. 12 shows two adjacent tubular rings having peaks out-of-phase with one another and coupled together with an arcuate connector 252. Both ends of connector 252 are connected to the outside radius of peaks 254, 256 on adjacent tubular rings. This configuration allows foreshortening during radial expansion. FIG. 13 shows an embodiment where a sigmoidal shaped connector 262 between adjacent out of-phase-tubular rings 260 is coupled to the inside radius of one peak 264 and the opposite end is coupled to the inside radius of a valley 266 thereby allowing lengthening during radial expansion. Various combinations of these connectors may be used to provide a prosthesis that lengthens on one side and shortens on an opposite side, as may be required when butting two prostheses against one another. FIG. 17 illustrates a chevron shaped connector 902 having two protrusions 904, 906 on opposite sides of the chevron thereby forming a stopping element. The chevron may expand outwardly, but motion is limited in compression and this is useful in reducing foreshortening during expansion of the prosthesis. FIG. 16 illustrates a chevron shaped connector 802 coupling adjacent tubular rings together. In addition to tapered struts 802, the connector is tapered such that stem 804 of connector 802 is wider than the rest of the axial portion of connector 802 and the apex of the chevron 806 is the widest portion of connector 802. By making connector 804 wider, it become stiffer and this helps reduce foreshortening during radial expansion as well as bending and bucking of the prosthesis.

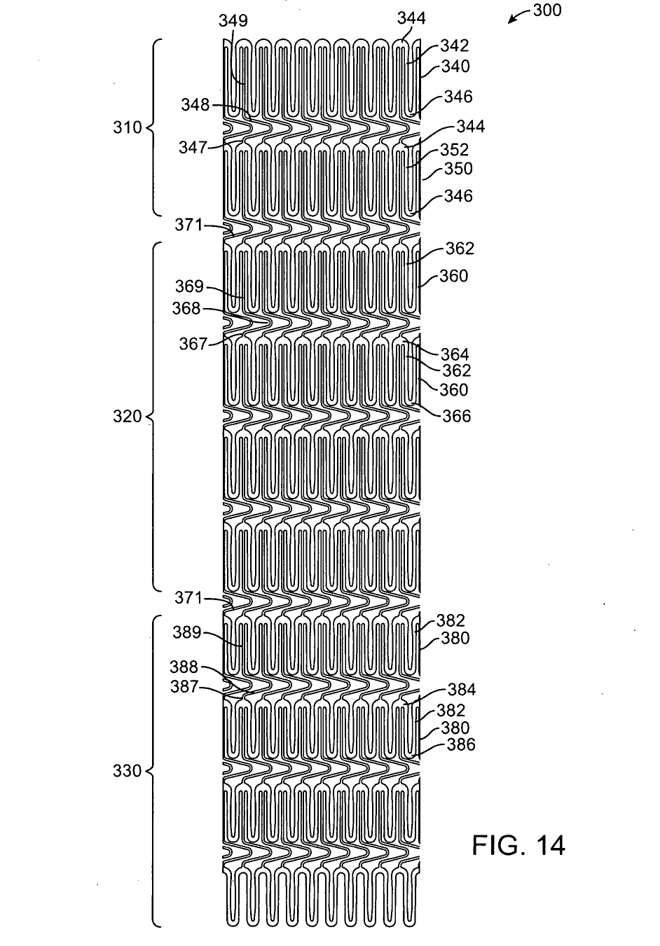

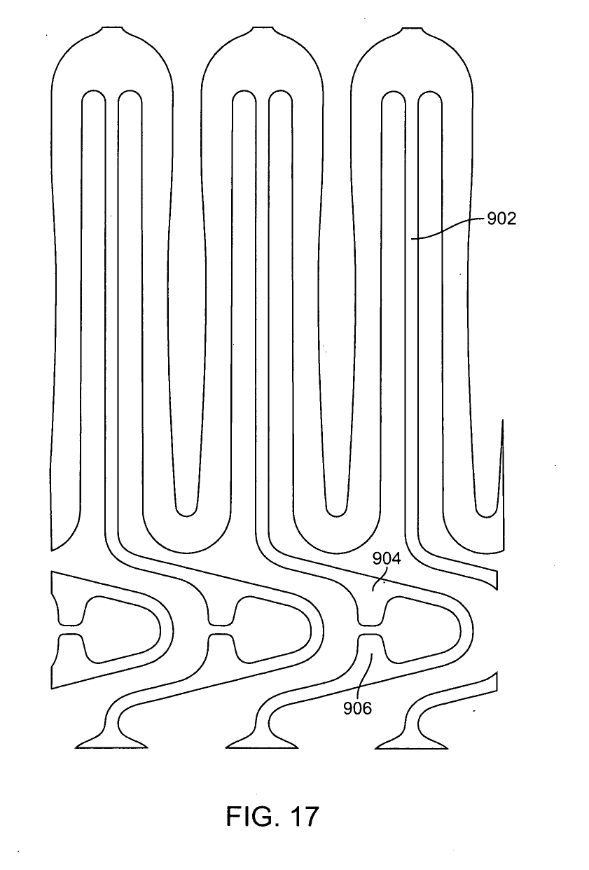

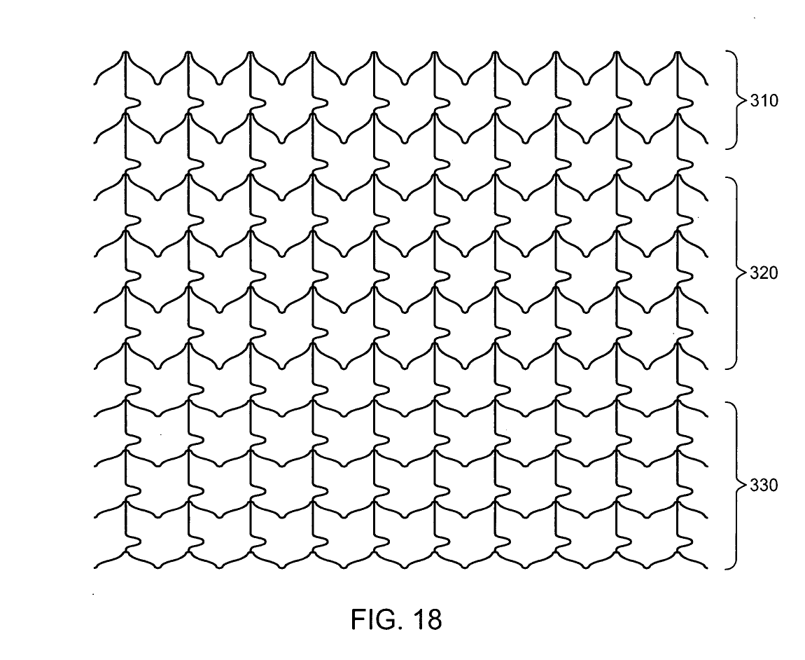

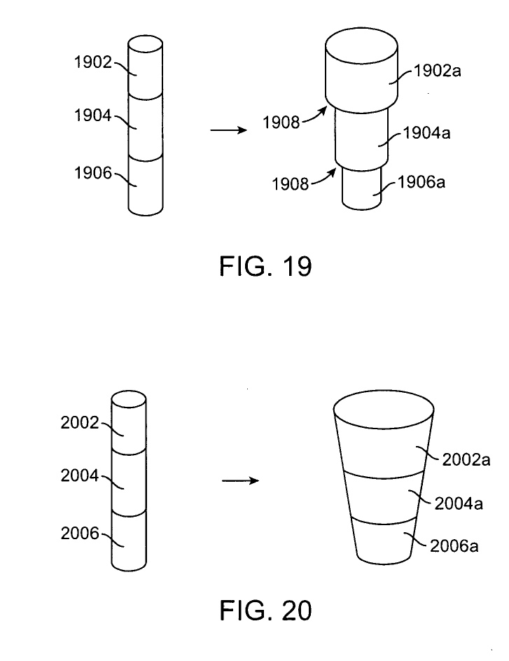

In the embodiment of FIG. 1, the transition between the proximal section and the central section as well as the transition between the central section and the distal section is somewhat abrupt. Strut length changes from one length to a shorter length from section to section. This may result in stepped regions when the prosthesis is expanded. FIG. 19 illustrates a schematic of a prosthesis similar to that of FIG. 1 in the contracted and expanded configurations. The prosthesis has a first region 1902, a second region 1906 and a central region 1904 each having similar peak and valley geometry as FIG. 1 but with each region having different struts length. When strut lengths from one region to the next change dramatically, stepped regions 1908 may form between the expanded first region 1902a, second region 1906a and the central region 1906b. It may be desirable to smooth the stepped regions out and provide a smoother, more tapered expanded prosthesis in order to conform to the anatomy better as well as providing a smoother path for blood flow. Thus, the prosthesis may be modified so that the change in strut length as well as the corresponding performance properties are more gradual and this can be achieved by altering strut length gradually over the course of several adjacent tubular rings and thus there will be no discrete dividing line between the proximal, central and distal sections. FIG. 20 illustrates a schematic of a prosthesis similar to FIG. 1 and having similar peak and valley geometry. The prosthesis of FIG. 20 has a first region 2002, a second region 2006 and a central region 2004 in the contracted configuration as well as the resulting smooth taper of the prosthesis when the first region 2002a, the second region 2006a and the central region 2004a have been expanded. FIG. 20 also includes similar peak and valley geometry as described above with respect to FIG. 1. FIG. 14 illustrates an exemplary embodiment of a tubular prosthesis having a more gradual transition between regions of the prosthesis which would produce a more tapered expanded shaped rather than a stepped shape. The prosthesis of FIG. 14 is seen flat and unrolled for ease in viewing.

The prosthesis 300 seen in FIG. 14 is similar to the embodiment of FIG. 1. It has three distinct regions, a proximal section 310, a central section 320 and a distal section 330. The major differences between the embodiments of FIG. 14 and FIG. 1 are the strut lengths in the proximal section 310 and the connector configurations. Prosthesis 300 is also an implantable endoframe that may be used in conjunction with a polymer or fabric cover in the treatment of aneurysms. The prosthesis 300 may be used alone or in combination with another prosthesis placed adjacent thereto.

Proximal section 310 comprises two adjacent tubular rings 340, 350, although the number of tubular rings may be more or less. Each tubular ring 340, 350 is comprised of a plurality of axially oriented struts 342, 352 that are coupled together to form a circumferential series of peaks 344 and valleys 346. Struts 342 are longer than struts 352, and struts 342, 352 are both longer than struts 362 in the central section 320 and struts 382 in the distal section 330 of the prosthesis 300. Thus tubular ring 340 requires less force to expand than the other rings in the in prosthesis 300, hence ring 340 will begin to expand first, followed by ring 350 and then the rings in the central 320 and distal section 330. Additionally, tubular ring 340 can expand to the largest diameter in the prosthesis 300 which is desirable since the proximal section 340 is often implanted in a region proximal to an aortic aneurysm which has the largest diameter (the proximal direction is closest to the patient's heart). Additionally, the proximal section 340 may be farther expanded in post-procedure adjustments (e.g. post procedure dilation or “tacking”). Strut lengths in the proximal section are similar to those of the proximal section struts in the embodiment of FIG. 1 previously described and thus the expansion ratios of prosthesis 300 are similar to those previously discussed with respect to the embodiment in FIG. 1.

The struts 342 and 352 are also tapered so that the thinnest portion of the strut is at the circumferential centerline of the tubular ring 340, 350. Width tapers outwardly as it extends to either a peak 344 or a valley 346. Struts 342 and 352 are therefore thickest at the apex of the peaks 344 and at the bottom of the valleys 346. Strut width is similar to that of the proximal section strut widths disclosed for the embodiment of FIG. 1.

A connector 348 having axially extending struts 347, 349 couple adjacent tubular rings 340, 350 together. Connector 348 is longer than conventional connectors which only traverse the gap between a peak and a valley on an adjacent tubular ring, thus the longer connector 348 is more flexible. In the embodiment of FIG. 14, the peaks 344 of rings 340, 350 are in-phase with one another, and thus connector 348 is coupled on both ends to a peak 344 in an adjacent tubular ring 340, 350. Connector 348 has a shorter axially extending strut 347 which is coupled to an outside radius of the apex one peak 344 while a longer axially extending strut 349 is coupled to an inside radius of peak 344 in the adjacent tubular ring 340. Connector 348 has a “v” or chevron shape and the axially oriented struts 347, 349 are substantially parallel to the longitudinal axis of the prosthesis 300. This configuration minimizes axial contraction of the proximal section 310 as the prosthesis 300 is radially expanded. Connector dimensions are similar to those described with respect to the proximal section connector dimensions for the embodiment in FIG. 1.

Central section 320 comprises four adjacent tubular rings 360, although this number may be varied as required. The proximal-most tubular ring of the central section 320 is coupled with the distal-most tubular ring of the proximal section 310 via a chevron shaped connector 371. One end of connector 371 is coupled to an outside radius of peak 364 and the opposite end of connector 371 is coupled with an inside radius of peak 364. Each tubular ring 360 is comprised of a plurality of axially oriented struts 362 coupled together to form a circumferential series of peaks 364 and valleys 366. The struts 362 in the central section 320 are shorter than the struts 342, 352 of the proximal section 3.10, but struts 362 are still longer than the struts 382 in the distal section 330. Thus, the central section 320 will begin to expand after the proximal section 310 begins to expand, but before the distal section 330 expands. In alternative embodiments, the struts 362 in the central section may have decreasing length from one tubular ring to the next. This further enhances the smooth transition along the prosthesis in the expanded configuration. Thus, the struts 362 in the ring closest to the proximal section are the longest and the struts 362 in the ring farthest away from the proximal section are the shortest and strut length in the rings between decrease proportionally. The length of struts 362 is optimized by tapering its width so that the thinnest portion of the strut is at the circumferential centerline of the tubular ring 360. Width tapers outwardly as it extends to either a peak 364 or a valley 366. Struts 362 are therefore thickest at the apex of the peaks 364 and at the bottom of the valleys 366. Strut dimensions are similar to those previously described in relation to the central section struts in the embodiment of FIG. 1.

A connector 368 having axially extending struts 367, 369 couple adjacent tubular rings 360 together. In the central section 320, peaks 364 on adjacent tubular rings are in-phase with one another, thus each peak 364 in one tubular ring 360 is coupled with a peak 364 in an adjacent tubular ring 360. Additionally, similar to the proximal section 310, one axially extending strut 369 is significantly longer than the other axially extending strut 369 such that the longer strut 369 is coupled to the inside radius of a peak 364 and the shorter strut 367 is coupled to the apex of the outside radius of a peak 364 in an adjacent tubular ring 360. The longer strut 369 is thin enough to nest between adjacent struts 362 on the adjacent tubular ring 360 and this helps reduce profile of the prosthesis 300 when in the crimped configuration. Because the axially extending strut 369 is not a primary load bearing member, it may be considerably thinner than the struts 362. Axially extending struts 367, 369 are also substantially parallel to the longitudinal axis of prosthesis 300 and the connector 368 is shaped like a “v” or a chevron. The arrangement of connectors 368 ensures that there is little or no relative motion between circumferential centerlines of adjacent tubular rings 360 and thus the central section 320 foreshortens a minimal amount radial expansion. Foreshortening in this embodiment is similar to that described above with respect to FIG. 1. Connector dimensions are similar to those previously disclosed with respect to the central section connector dimensions of FIG. 1.

FIG. 14 also shows the distal region 320 of prosthesis 300. Distal section 330 is coupled to the central section via chevron shaped connectors 371. Connector 371 is coupled to an outside radius of the apex of peak 384 and the opposite end of connector 371 is coupled to an inside radius of peak 364. Distal section 330 comprises four tubular rings 380, although this number may be modified as required. Each tubular ring 380 comprises a plurality of axially oriented struts 382 coupled together to form a circumferential series of peaks 384 and valleys 386. The struts 382 in the distal section 330 are the shortest struts in prosthesis 300 as compared with struts 362 in the central section 320 and struts 352 and 342 in the proximal section 310. Because struts 382 are the shortest, distal section 330 will be the last section of prosthesis 300 to radially expand during deployment. Additionally, shorter struts in the distal section 330 help to ensure more uniform expansion over the circumference of the distal section 330 which is often placed distal to an aneurysm, in or near the iliac arteries where diameter is considerably smaller as compared with the diameter of proximal section 310 which may be placed in the aorta. Using longer struts in the distal section 330 would result in only a few of the struts opening up to match vessel diameter and this effect can be further exacerbated if the expandable balloon which is used to expand prosthesis 300 is not folded down uniformly, as previously discussed above. Thus, using shorter struts in the distal section 330 reduces the sensitivity of the distal section 330 to uneven expansion. In alternative embodiments, strut length may also vary from ring to ring within the distal region to provide an even better, smoother transition when the prosthesis is expanded. Thus, struts in the ring closest to the central region are the longest and struts in the ring farthest away from the central region are the shortest, with intermediate length struts in between. The struts 382 are also tapered similarly to the struts of the proximal 310 and central 320 sections of the prosthesis 300. Struts 382 are tapered so that the thinnest portion of the strut is at the circumferential centerline of the tubular ring 380. Width tapers outwardly as it extends to either a peak 384 or a valley 386. Struts 382 are therefore thickest at the apex of the peaks 384 and at the bottom of the valleys 386. The dimensions of the struts 382 are similar to the distal region struts illustrated in FIG. 1. Strut thickness and aspect ratio in all regions of the embodiment of FIG. 14 are similar to those previously disclosed with respect to the embodiment of FIG. 1. Other performance characteristics of the embodiment illustrated in FIG. 14 such as foreshortening, kinking resistance, etc. are also similar to those described in reference to FIG. 1.

Additionally, due to the shorter strut 382 length in the distal section 330, the number of rings per linear length, or pitch, increases relative to the other sections of the prosthesis 300. This feature has the added benefit of allowing the distal section 330 to accommodate tighter bends in the blood vessels without kinking, as is often seen in the iliac arteries.

Connector 388 has axially extending struts 387, 389 and couples adjacent tubular rings 380 together. In the distal section 330, peaks 384 on adjacent tubular rings are in-phase with one another, thus each peak 384 in one tubular ring 380 is coupled with a peak 380 in an adjacent tubular ring 380. Connector 388 has one axially extending strut 389 which is significantly longer than the other axially extending strut 387, and the longer strut 389 is coupled to an inside radius of peak 384 while the shorter strut 387 is coupled to the outside radius of peak 384 in an adjacent tubular ring 380. Similar to the central section 320, the longer strut 389 is thin enough to nest between adjacent struts 382 in one tubular ring 380 and this helps reduce the profile of the prosthesis 300 in the crimped configuration. Also, the axially extending strut 389 is not a primary load bearing member thus it may be considerably thinner than struts 382. Axially extending struts 387, 389 are also substantially parallel to the longitudinal axis of prosthesis 300 and connector 388 is shaped like a “v” or chevron. The arrangement of connectors 388 ensures that there is little or no relative motion between circumferential centerlines of adjacent tubular rings 380 and thus foreshortening of the distal section 330 is also similar to the rest of the prosthesis. Foreshortening is thus about 2% or less during radial expansion of the prosthesis 300. Connector dimensions are similar to the distal region connector dimensions previously disclosed for the embodiment in FIG. 1.

FIG. 18 illustrates the proximal 310, central 320 and distal sections 330 of prosthesis 300 in the expanded state.

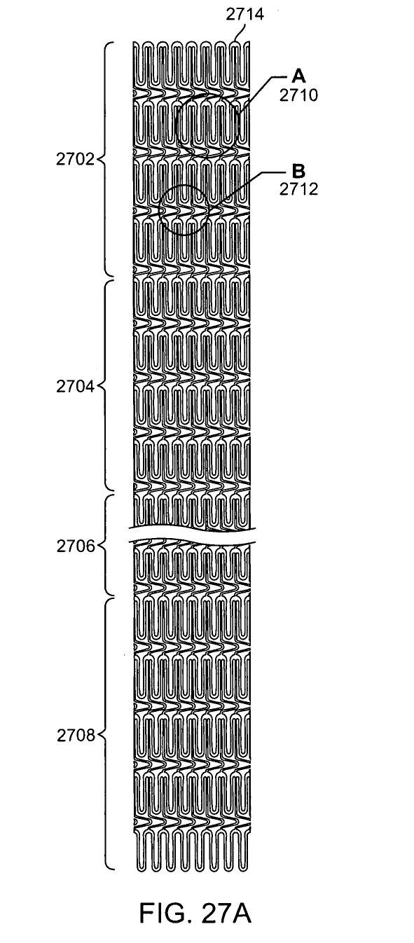

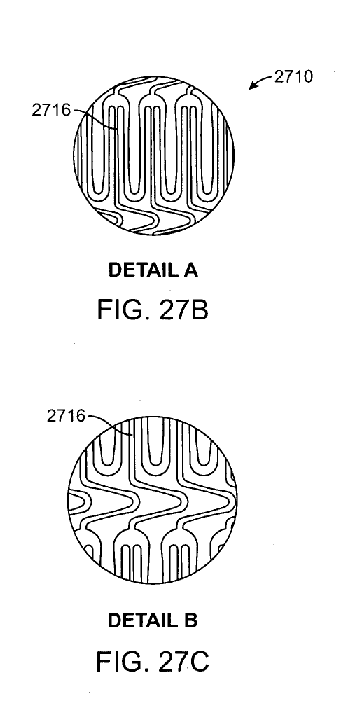

FIGS. 27A-27C illustrate yet another exemplary embodiment of a prosthesis having axially variable characteristics. The embodiment illustrated in FIG. 27A is similar to the previous embodiment illustrated in FIG. 14 above, with the major difference being the number of peaks per ring and also the connector geometry. Other aspects of the embodiment in FIG. 27A are generally the same as described above in FIG. 14. For example, the rings are formed from a plurality of struts connected together to form a series of peaks and valleys with adjacent rings coupled together with a connector. In FIG. 27A, the prosthesis is comprised of four regions, a neck region 2702, a taper region 2704, a body region 2706 and a flare region 2708. The four regions allow a smooth transition along the prosthesis when radially expanded in a patient, often in an aneurysm. Unlike the embodiment of FIG. 14 which has ten peaks per ring, in FIG. 27A, each ring has eight peaks 2714. One of skill in the art will of course appreciate that the number of peaks per ring can be varied and in other exemplary embodiments twelve peaks may be used per ring. The struts in region 2710 which form the peaks and valleys in the second ring of the neck region section are highlighted in greater detail in FIG. 27B. Similarly the connectors in region 2712 coupling adjacent rings together are highlighted in greater detail in FIG. 27C. FIG. 27C also illustrates that the connectors 2716 in this embodiment are tapered toward their connection point with an inner radius of a peak.

While the exemplary embodiment of FIG. 14 describes a “v” shaped or chevron shaped connector, any of the connectors and connection points previously described may be used with this embodiment. Additionally, tubular rings may be arranged so that they are in-phase or out-of-phase with one another as desired. Thus any combination of the features described herein may be utilized in a prosthesis having axially variable properties. The prostheses disclosed herein may be self-expanding or they may be balloon expandable. Often, self-expanding prostheses are fabricated from nickel titanium alloys such as Nitinol while balloon expandable prostheses are often composed of stainless steel, cobalt chromium alloys and the like. Polymers may also be used to fabricate the prostheses which are typically manufactured by laser cutting or EDM (electrical discharge machining) tubing or photochemically etching flat sheet stock. The etched sheet is then rolled into a tube and the opposite ends are welded together. Additionally, therapeutic agents such as heparin may be carried by the prosthesis and controllably released in order to reduce the risk of thrombosis after implantation.

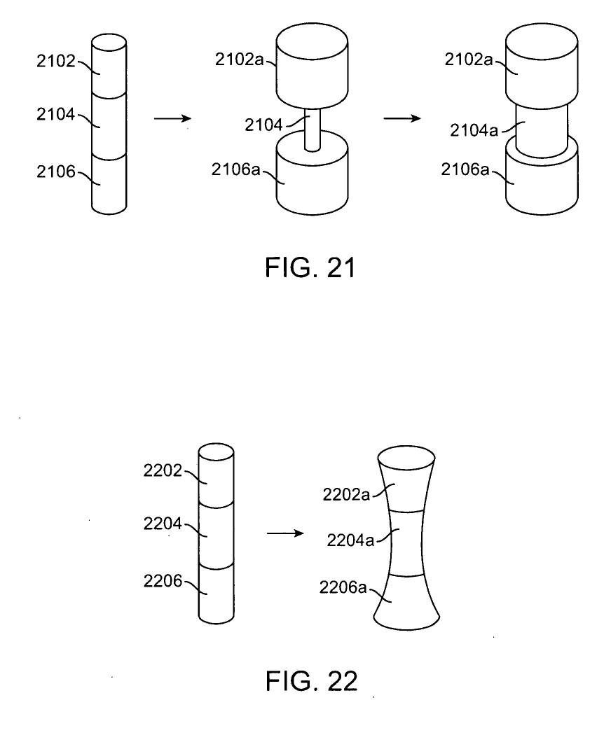

Therefore, varying strut length from section to section of a prosthesis produces a prosthesis having axially variable properties. Diameter and order of expansion of the prosthesis may then be controlled. Other exemplary embodiments include but are not limited to the following. FIG. 21 schematically illustrates a three section prosthesis. In the collapsed configuration, the prosthesis have a first section 2102, a central section 2104 and a second section 2106. The struts in the central section 2104 are shorter than the struts in the first or second sections 2102, 2106 such that upon expansion, the first and second sections radially expand to their expanded diameters 2102a, 2106a before the central section. The central section 2104a expands afterwards. Strut length in the first and second sections may be varied in order to obtain a desired expanded configuration. In FIG. 21, the struts in the first and second sections 2102, 2106 are approximately the same length, thus the expanded diameter of both sections 2102a, 2106a are about the same. However, the strut length in the second section 2106 could be shorter than the struts in first section 2102 so that the second section expands to a smaller diameter than the first section. Other variations are also possible.

FIG. 22 illustrates another three section prosthesis in the unexpanded configuration having a first section 2202, a central section 2204 and a second section 2206. The first and second sections 2202, 2206 could have multiple rings with strut length gradually increasing toward each end such that in the expanded configuration, the prosthesis has one or both ends flared. Flared ends 2202a and 2206a allow the prosthesis to more accurately match the patient's anatomy as well as provide a smooth transition into and out of the prosthesis. This could facilitate docking with an extension prosthesis such as might be used in an iliac artery. The flared region also helps to prevent plaque extrusion and embolization from an aneurysm Central section 2204a may also have a gradual flare or taper so that it provides a smooth transition between the first and second sections 2202a, 2206a.

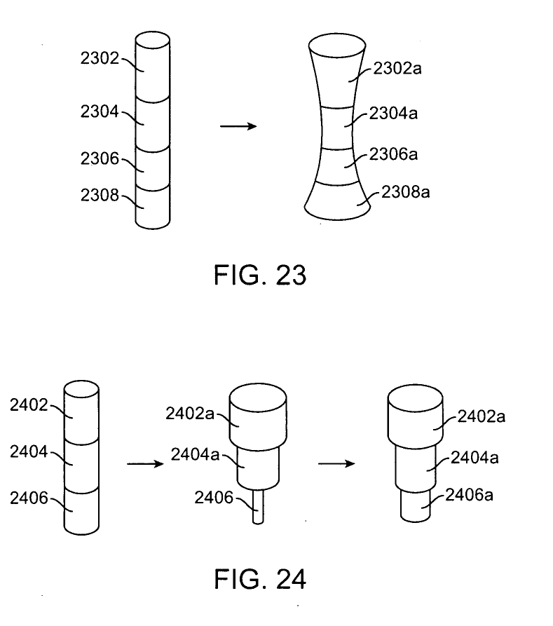

FIG. 23 illustrates yet another embodiment, this time a four section prosthesis. In the unexpanded configuration, the four sections 2302, 2304, 2306 and 2308 generally have the same unexpanded diameter. Varying the strut length in each section allows the expanded configuration to be controlled. In this embodiment for example, struts in the first section and the fourth section may be larger than the two remaining sections 2304, 2306 such that such that in the expanded configuration, flared ends 2302a and 2308a may be obtained. The two remaining sections 2304a, 2306a provide a smooth transition between flared ends. One of skill in the art will appreciate that any number of sections may be used in order to provide a desired prosthesis length having desired expansion characteristics.

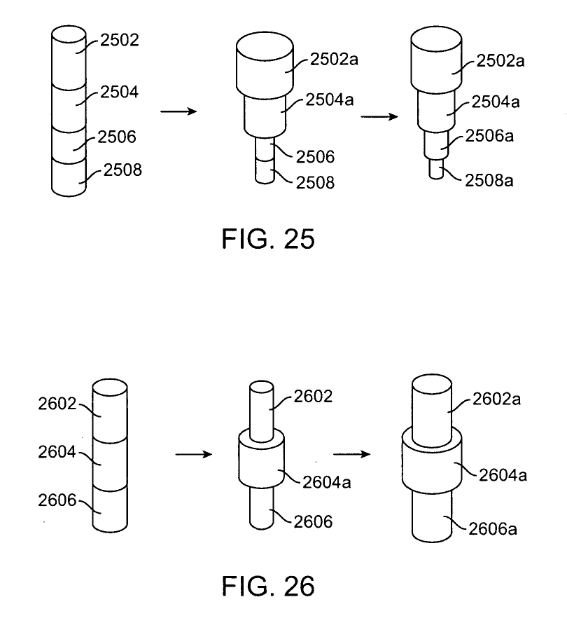

In still another embodiment, a three section prosthesis may be produced by varying strut length such that one end opens last. This is advantageous since it allows the prosthesis to hold onto or hug a balloon during delivery, preventing unwanted ejection or other movements of the prosthesis relative to the delivery catheter. In FIG. 24, the unexpanded prosthesis has a first section 2402, a central section 2404 and a second section 2406. Upon radially expansion, the first section 2402a and the central section 2404a open first. The second section opens last 2406a. A four section variation on this is illustrated in FIG. 25. In FIG. 25, the prosthesis in the contracted configuration has first, second, third and fourth sections 2502, 2504, 2506, 2508. The first two sections 2502, 2504 have struts longer than the other two sections 2506, 2508, thus the first two sections 2502a, 2504b expand first, followed by the last two sections 2506a, 2508a. This embodiment shows the prosthesis tapering from the first section 2502a to the last section 2508a, but one of skill in the art will appreciate that strut length in each section may be adjusted to any number of other configurations including flaring one or both ends, a central section larger than the ends, etc.

FIG. 26 shows another exemplary embodiment of an axially variable prosthesis. In FIG. 26, the contracted prosthesis has a first section 2602, a second section 2606 and a central section 2604. The struts in the central section 2604 are longer than the two other sections 2602, 2606, thus upon radial expansion, the central section expands first 2604a, followed by the two other sections, 2602a, 2606a.

The exemplary embodiments illustrated in FIGS. 19-26 are schematic diagrams. Each of the sections in these embodiments may have any of the multiple ring, strut and connector geometries previously described above, as well as other geometries known to those skilled in the art. Thus any of the features disclosed herein may be used to create a prosthesis having axially variable characteristics.

While the embodiments disclosed above relied primarily on strut length to control expansion order and diameter of the prosthesis, one of skill in the art will also appreciate that a number of other properties of the prosthesis may be varied in order to obtain similar results. For example, different geometries and material properties may be varied. Some of these include but are not limited to strut length, strut width, strut thickness, number of struts per cell, connector radius, connector thickness, connector geometry, material temper, material strength, and combinations thereof. Thus, a prosthesis with axially variable characteristics may be fabricated by producing a first section of a prosthesis with one set of these properties and then producing a second section of the prosthesis with a second set of these properties. Additional sections of the prosthesis may also be produced to obtain a longer prosthesis with the same or different characteristics of the other sections. Prostheses having ten or more sections may be produced, although preferably the prosthesis has 5-7 sections and even more preferably 3-4 sections. Fabricating techniques often involve laser cutting, electrical discharge machining or photochemical etching of tubing or flat sheet of metal, polymers or other materials.

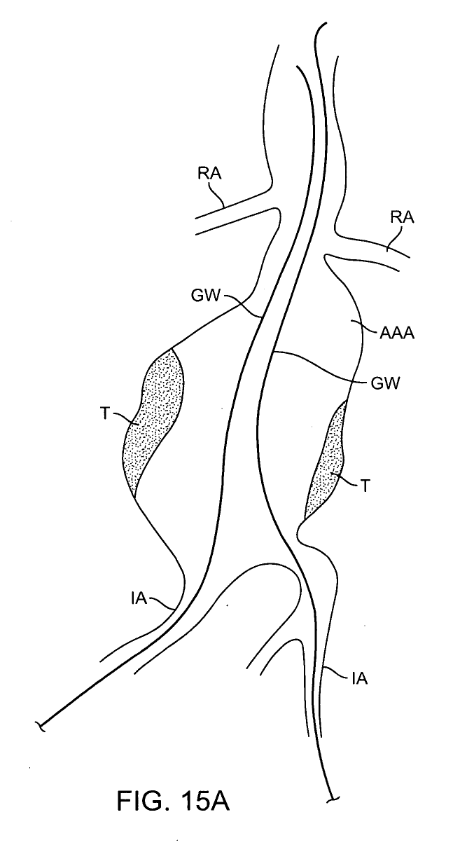

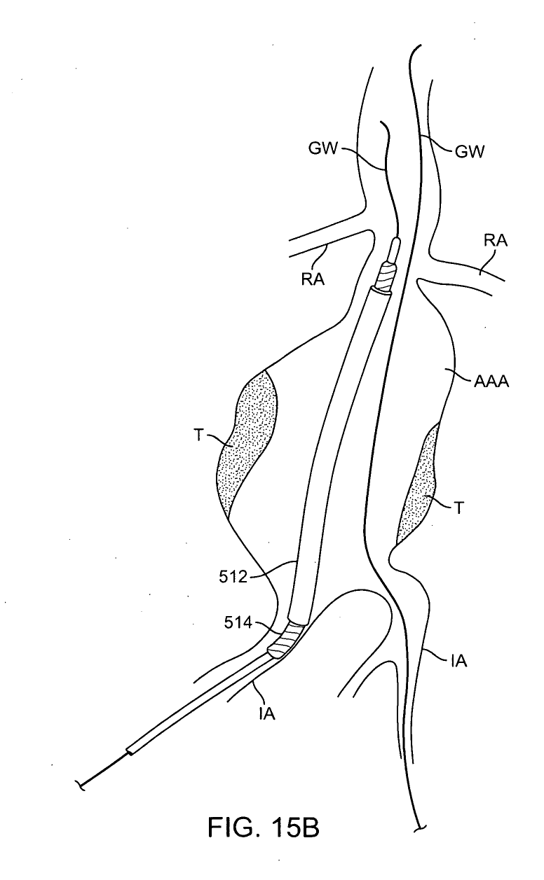

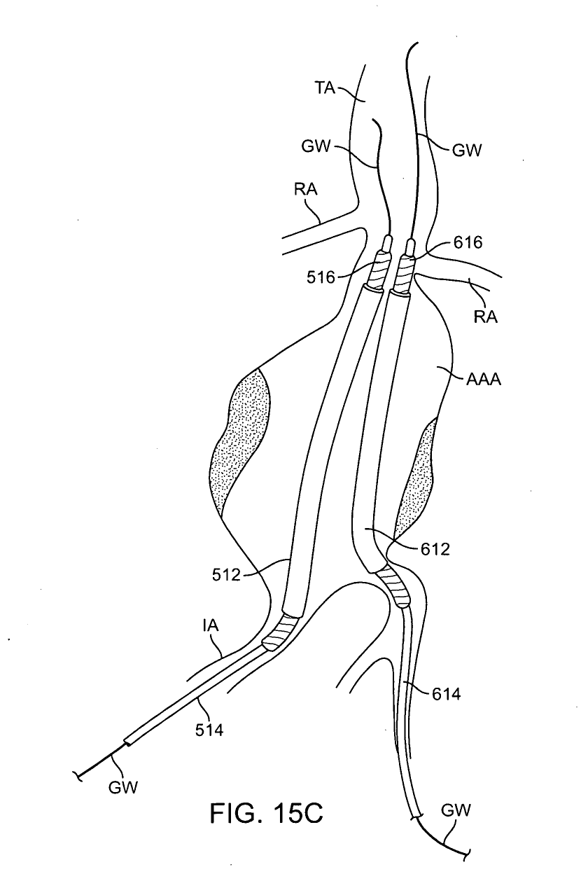

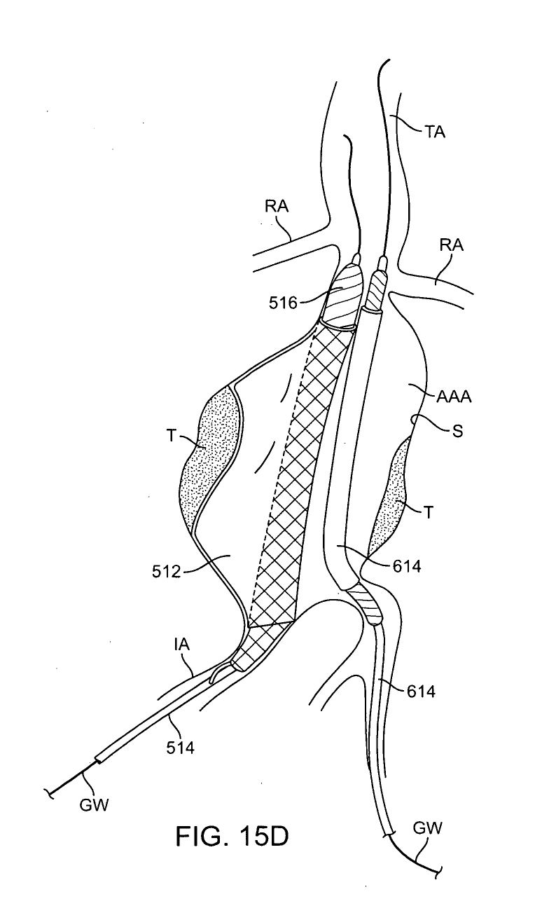

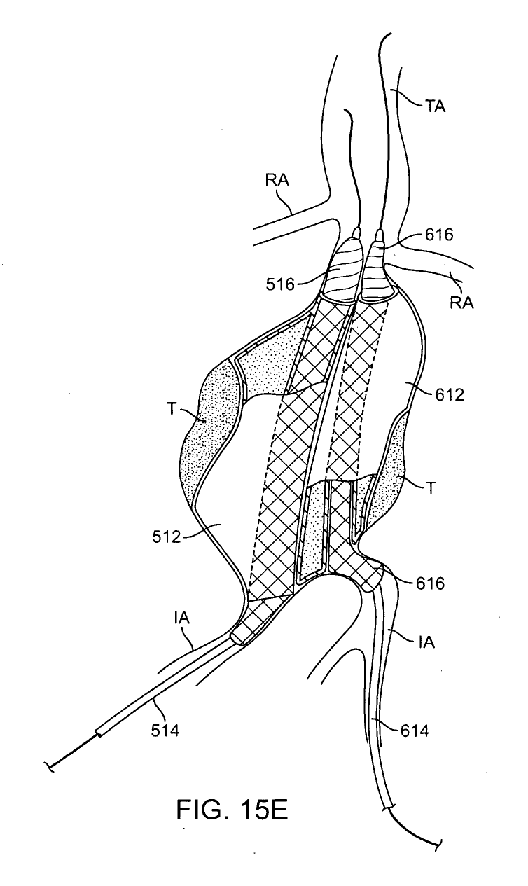

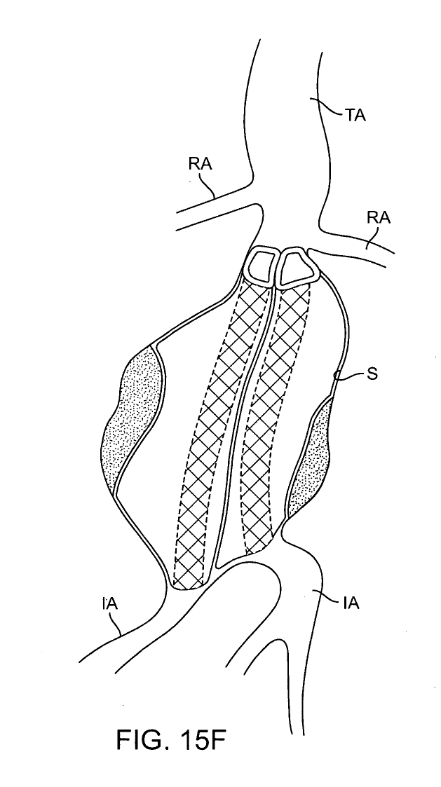

Any of the prostheses described herein may be used with a fabric or polymer cover such as an ePTFE double walled fillable structure to treat an aneurysm. Double walled fillable structures are disclosed in U.S. Patent Publication No. 2006/0025853, the entire contents of which are incorporated herein by reference. FIGS. 15A-15F illustrate an exemplary method of treating an aneurysm with two endoframes such as those described herein. Each endoframe is combined with a double walled fillable structure. FIG. 15A illustrates the anatomy of an infrarenal abdominal aortic aneurysm (AAA) that is disposed between the renal arteries RA and the iliac arteries IA. The aneurismal sac may have regions of mural thrombus T over portions of its inner surface.

In treating an infrarenal abdominal aortic aneurysm a pair of endoframes is combined with filling structures to form prostheses 512 and 612, and a pair of guidewires (GW) will first be introduced, one from each of the iliac arteries (IA), as illustrated in FIG. 1SA. The first delivery catheter 514 will then be positioned over one of the guidewires to position the endoframe with double-walled filling structure 512 across the aortic aneurysm (AAA), as illustrated in FIG. 15B. The second delivery catheter 614 is then delivered over the other guidewire (GW) to position the second endoframe with filling structure 612 adjacent to the first structure 512 within the aneurysm (AAA), as illustrated in FIG. 15C. Typically, a protective sheath (not illustrated) is retracted and one of the prostheses 512 or 612 and associated balloons 516 or 616 will be expanded first, followed by the other of the prostheses and balloon, as illustrated in FIG. 15D where the endoframe and filling structure 512 along with balloon 516 are inflated to fill generally half of the aneurismal volume, as illustrated in FIG. 15D. Radially expanding the endoframe helps create a lumen for blood flow therethrough as well as anchoring the prosthesis in and around the aneurysm. Thus, the tubular body of the endoframe upon expansion creates an endoframe or endoskeleton support structure for one or more new lumens to be formed by surrounding the endoframe with a fillable endograft containment system which is filled with a polymer that cures in situ. The fillable structure also helps to anchor the device into the aneurismal sac and also helps prevent lateral movement of the prosthesis which reduces the chance of leaks forming later on. The filling structure 512 will be expanded to occupy only about one-half of the aneurismal volume. After the first filling structure 512 has been filled with a fluid such as polyethylene glycol or other materials disclosed in U.S. Patent Publication No. 2006/0025853, the second endoframe and filling structure 612 may be filled, as illustrated in FIG. 15E. The upper ends of the balloons 516 and 616 will conform the tubular lumens of the filling structures against the walls of the aorta as well as against each other, while the lower ends of the balloons 516 and 616 will conform the tubular lumens into the respective iliac (IA) artery. In other embodiments, both filling structures surrounding the expanded endoframes may be filled simultaneously. In addition, either or both of the filling structures surrounding the expanded endoframes may be filled with the balloons in the inflated state or deflated state to allow perfusion and conformance or apposition of the endoframes to the tortuosity of the vasculature or the anatomy of the aortic neck and iliac arteries. The tubular endoframe in the expanded configuration should be strong enough such that it maintains at least 50% of its expanded diameter when an external differential radial pressure of about 60 mm of Hg to about 1000 mm of Hg is applied. Similarly, when two endoprostheses are used together, the proximal section of each should similarly be able to withstand about 60 mm of Hg to about 1000 mm of Hg of externally applied differential radial pressure without more than a 50% reduction in expanded diameter. Thus the endoframe enables or maintains blood perfusion during filling and curing of the filling structures.

After expanding the endoframe and filling the filling structures 512 and 612 as illustrated in FIG. 15E, the filling materials or medium will be cured or otherwise hardened, and the delivery catheters 514 and 614 removed, respectively. The hardened filling structures will then provide a pair of tubular lumens opening from the aorta beneath the renal arteries to the right and left iliac arteries, as shown in broken line in FIG. 15F. The ability of the filling structures 512 and 612 to conform to the inner surface (S) of the aneurysm, as shown in FIG. 15F, helps assure that the structures will remain immobilized within the aneurysm with little or no migration. Immobilization of the filling structures 512 and 612 may be further enhanced by providing any of the surface features described in U.S. Patent Publication No. 2006/0025853, previously incorporated herein by reference.

While the above is a complete description of the preferred embodiments of the invention, various alternatives, modifications, and equivalents may be used. Therefore, the above description should not be taken as limiting in scope of the invention which is defined by the appended claims.

What is claimed is:

1. A prosthesis comprising:
a tubular body expandable from a contracted configuration to a radially expanded configuration, the tubular body having a total length and comprising a first section, a second section and a central section disposed therebetween,
wherein the total length of the tubular body in the expanded configuration is at least 95% of the total length of the tubular body in the contracted configuration, and
wherein the first section comprises a plurality of tubular rings, each ring comprising a plurality of struts having a length, the struts of the first section coupled together to form a circumferential series of peaks and valleys and a connector coupling adjacent tubular rings together, and
wherein the second section comprises a plurality of tubular rings, each ring comprising a plurality of struts having a length and coupled together to form a circumferential series of peaks and valleys and a connector coupling adjacent tubular rings together, and
wherein the central section comprises a plurality of tubular rings, each ring comprising a plurality of struts having a length and coupled together to form a circumferential series of peaks and valleys and a connector coupling adjacent tubular rings together, the length of the central section struts being different than the length of the first and second section struts, and
wherein the central section is coupled with the first and second sections.

2. The prosthesis of claim 1, wherein the length of the first section struts is greater than the length of the second section struts and the length of the central section struts, and
wherein the length of the central section struts is greater than the length of the second section struts,
wherein the first section has a diameter in the expanded configuration greater than a diameter of the second and central sections in the expanded configuration, and
wherein the diameter of the central section in the expanded configuration is greater than the diameter of the second section in the expanded configuration, and
wherein the first section is adapted to radially expand first, followed by radial expansion of the central section which is followed by radial expansion of the second section.

3. The prosthesis of claim 2, wherein the tubular body comprises a stepped region between either an outer surface of the first section in the expanded configuration and an outer surface of the central section in the expanded configuration or between an outer surface of the central section in the expanded configuration and an outer surface of the second section in the expanded configuration.

4. The prosthesis of claim 1, wherein the first section comprises a first ring and a second ring, and
wherein the first ring comprises struts having the first section strut length and the second ring comprises struts having a length less than the first section strut length, the second ring strut length also greater than the second section strut length and the central section strut length, and
wherein the tubular body in the expanded configuration tapers substantially uniformly from the first section to the central section and the second section.

5. The prosthesis of claim 1, wherein the central section strut length is less than both the first section strut length and the second section strut length, and wherein the central section is adapted to radially expand after both the first section and the second section radially expand.

6. The prosthesis of claim 1, wherein the first section strut length and the second section strut length are greater than the central section strut length, and wherein the first section and the second section are adapted to radially expand before the central section radially expands.

7. The prosthesis of claim 1, wherein the first section comprises a first ring and a second ring, the second ring closer to the central section than the first ring, and
wherein the first ring comprises struts having the first section strut length and the second ring comprises struts having a length less than the first section strut length, and
wherein the tubular prosthesis in the expanded configuration comprises a first flared end, the first flared end comprising the first and second rings, the first ring having an expanded diameter larger than an expanded diameter of the second ring.

8. The prosthesis of claim 7, wherein the second section comprises a first ring and a second ring, the second ring being closer to the central section than the first ring, and
wherein the first ring of the second section comprises struts having the second section strut length and the second ring of the second section comprises struts having a length less than the second section strut length, and
wherein the tubular prosthesis in the expanded configuration comprises a second flared end opposite the first flared end, the second flared end comprising the first and second rings of the second section, the first ring of the second section having an expanded diameter larger than an expanded diameter of the second ring in the second section.

9. The prosthesis of claim 7, further comprising a fourth section, the fourth section disposed between the first and central sections or between the central and second sections,
wherein the fourth section comprises a plurality of tubular rings, each ring comprising a plurality of struts having a length, the struts of the fourth section coupled together to form a circumferential series of peaks and valleys and a connector coupling adjacent tubular rings together.

10. The prosthesis of claim 1, wherein the second section strut length is less than both the first section strut length and the central section strut length, and wherein the second section is adapted to radially expand after the first section and the central section radially expand.

11. The prosthesis of claim 1, further comprising a fourth section, the fourth section disposed between the central section and the second section,
wherein the fourth section comprises a plurality of tubular rings, each ring comprising a plurality of struts having a length, the struts of the fourth section coupled together to form a circumferential series of peaks and valleys and a connector coupling adjacent tubular rings together, and
wherein the strut length in the second section and the fourth section is less than the strut length in the first section and the central section, and
wherein the first section and the central section are adapted to radially expand prior to radial expansion of the second and fourth sections.

12. The prosthesis of claim 1, wherein the central section strut length is greater than the first section strut length and the second section strut length, and wherein the central section is adapted to radially expand prior to radial expansion of both the first and second sections.

13. The prosthesis of claim 1, wherein the tubular body has a first diameter in the contracted configuration and a second diameter in the expanded configuration and wherein the ratio of the second diameter to the first diameter is greater than 1 and less than about 15.

14. The prosthesis of claim 1, wherein the tubular body is balloon expandable.

15. The tubular prosthesis of claim 1, wherein the tubular body has a diameter in the radially expanded configuration and wherein the tubular body maintains at least 50% of the radially expanded diameter when an externally applied differential radial pressure of between about 60 to about 1000 mm of Hg is applied thereto.

16. The prosthesis of claim 1, wherein in the first section, the peaks of a first tubular ring are out-of-phase with the peaks in an adjacent tubular ring.

17. The prosthesis of claim 1, wherein the first section comprises two tubular rings.

18. The prosthesis of claim 17, wherein the two tubular rings comprise a first tubular ring and a second tubular ring adjacent thereto, and
wherein a first connector couples the first tubular ring with the second tubular ring, and wherein one end of the first connector is coupled with a valley of the second tubular ring, and
wherein a second connector couples the second tubular ring with an adjacent tubular ring, and wherein one end of the second connector coupled with an inside radius of a peak of the second tubular ring.

19. The prosthesis of claim 17, wherein the two tubular rings comprise a first tubular ring and a second tubular ring adjacent thereto, and
wherein a first connector having first and second ends couples the first tubular ring with the second tubular ring, and
wherein the first end is coupled with an inside radius of a peak in the first tubular ring and the second end is coupled with a valley in the second ring.

20. The prosthesis of claim 1, wherein the connector in the first section has a first end coupled to a valley in a first tubular ring and a second end coupled to either a peak or a valley in an adjacent tubular ring.

21. The prosthesis of claim 20, wherein the second end is coupled to an inside radius of a peak in the adjacent tubular ring.

22. The prosthesis of claim 1, wherein the connector in the first section comprises a region having a chevron-like shape.

23. The prosthesis of claim 22, wherein the connector allows the prosthesis to be formed into a curve having a radius of 0.2 inches or more without forming a kink therein.

24. The prosthesis of claim 23, wherein the kink comprises a collapsed region of the tubular prosthesis having a diameter in the expanded configuration less than 50% of the diameter of the tubular prosthesis in the expanded configuration.

25. The prosthesis of claim 23, wherein the kink comprises a collapsed region of the tubular prosthesis having a cross-sectional area less than 50% of an uncollapsed cross-sectional area of the prosthesis.

26. The prosthesis of claim 1, wherein in the first section the struts have a width and the peaks have a width greater than the strut width.

27. The prosthesis of claim 1, wherein in the first section the connector has a width and the struts have a width wider than the connector width.

28. The prosthesis of claim 1, wherein the struts of the first section have a width and the width varies along a longitudinal axis of the strut.

29. The prosthesis of claim 1, wherein the struts of the first section have a first end, a second end opposite thereof and a central region therebetween and wherein strut width increases from the central region of the strut to either the first end or the second end.

30. The prosthesis of claim 1, wherein the struts of the first section have a width and the width is greatest at the peaks.

31. The prosthesis of claim 1, wherein the central section strut length is less than the first section strut length.

32. The prosthesis of claim 1, wherein in the central section, the peaks of a first tubular ring are in-phase with the peaks in an adjacent tubular ring.

33. The prosthesis of claim 1, wherein the central section comprises at least four tubular rings.

34. The prosthesis of claim 1, wherein the connector in the central section has a first end coupled to a peak in a first tubular ring and a second end coupled to either a peak or a valley in an adjacent tubular ring.

35. The prosthesis of claim 34, wherein the first end is coupled to an inside radius of the peak.

36. The prosthesis of claim 1, wherein the connector in the central section comprises a region having a chevron-like shape.

37. The prosthesis of claim 36, wherein the connector allows the prosthesis to be formed into a curve having a radius of 0.2 inches or more without forming a kink therein.

38. The prosthesis of claim 37, wherein the kink comprises a collapsed region of the tubular prosthesis having a diameter in the expanded configuration less than 50% of the diameter of the tubular prosthesis in the expanded configuration

39. The prosthesis of claim 37, wherein the kink comprises a collapsed region of the tubular prosthesis having a cross-sectional area less than 50% of an uncollapsed cross-sectional area of the prosthesis.

40. The prosthesis of claim 1, wherein in the central section the struts have a width and the peaks have a width wider than the strut width.

41. The prosthesis of claim 1, wherein in the central section the connector has a width and the struts have a width wider than the connector width.

42. The prosthesis of claim 1, wherein the struts of the central section have a width and the width varies along a longitudinal axis of the strut.

43. The prosthesis of claim 1, wherein the struts of the central section have a first end, a second end opposite thereof and a central region therebetween and wherein strut width increases from the central region of the strut to either the first end or the second end.

44. The prosthesis of claim 1, wherein the struts of the central section have a width and the width is greatest at the peaks.

45. The prosthesis of claim 1, wherein the second section strut length is less than the central section strut length.

46. The prosthesis of claim 1, wherein in the second section the pitch of the tubular rings is greater than the pitch of tubular rings in the first or central sections.

47. The prosthesis of claim 1, wherein in the second section, the peaks of a first tubular ring are in-phase with the peaks in an adjacent tubular ring.

48. The prosthesis of claim 1, wherein the second section comprises four tubular rings.

49. The prosthesis of claim 1, wherein the connector in the second section has a first end coupled to a peak in a first tubular ring and a second end coupled to either a peak or a valley in an adjacent tubular ring.

50. The prosthesis of claim 49, wherein the second end is coupled to an inside radius of a peak in the adjacent tubular ring.

51. The prosthesis of claim 1, wherein the connector in the second section has a first end coupled to a valley in a first tubular ring and a second end coupled to either a peak or a valley in an adjacent tubular ring.

52. The prosthesis of claim 1, wherein the connector in the second section comprises a region having a chevron-like shape.

53. The prosthesis of claim 52, wherein the connector allows the prosthesis to be formed into a curve having a radius of 0.2 inches or more without forming a kink therein.

54. The prosthesis of claim 53, wherein the kink comprises a collapsed region of the tubular prosthesis having a diameter in the expanded configuration less than 50% of the diameter of the tubular prosthesis in the expanded configuration.

55. The prosthesis of claim 53, wherein the kink comprises a collapsed region of the tubular prosthesis having a cross-sectional area less than 50% of an uncollapsed cross-sectional area of the prosthesis.

56. The prosthesis of claim 1, wherein in the second section the struts have a width and the peaks have a width wider than the strut width.

57. The prosthesis of claim 1, wherein in the second section the connector has a width and the struts has a width wider than the connector width.

58. The prosthesis of claim 1, wherein the struts of the second section have a width and the width varies along a longitudinal axis of the strut.

59. The prosthesis of claim 1, wherein the struts of the second section have a first end, a second end opposite thereof and a central region therebetween and wherein strut width increases from the central region of the strut to either the first end or the second end.

60. The prosthesis of claim 1, wherein the struts of the second section have a width and the width is greatest at the peaks.

61. The prosthesis of claim 1, further comprising a cover coupled to at least a portion of the tubular body.

62. The prosthesis of claim 61, wherein the cover comprises an inflatable member.

63. The prosthesis of claim 61, wherein the cover comprises a polymer.

64. The prosthesis of claim 61, wherein the cover comprises ePTFE.

65. The prosthesis of claim 1, wherein at least one of the connectors in the first, second or central sections comprises an elongate tapered strut.

66. The prosthesis of claim 1, wherein at least one of the connectors in the first, second or central sections comprises a strut having a chevron-like shape, wherein the widest width of the strut is at the apex of the chevron.

67. The prosthesis of claim 66, wherein the connector allows the prosthesis to be formed into a curve having a radius of 0.2 inches or more without forming a kink therein.

68. The prosthesis of claim 67, wherein the kink comprises a collapsed region of the tubular prosthesis having a diameter in the expanded configuration less than 50% of the diameter of the tubular prosthesis in the expanded configuration.

69. The prosthesis of claim 67, wherein the kink comprises a collapsed region of the tubular prosthesis having a cross-sectional area less than 50% of an uncollapsed cross-sectional area of the prosthesis.

70. The prosthesis of claim 1, wherein at least one of the connectors in the first, second or central sections comprises a strut forming a chevron-like pattern, wherein the strut further comprises a stopping element adapted to prevent the chevron from collapsing.

71. The prosthesis of claim 70, wherein the stopping element comprises a first raised region of the strut and a second raised region of the strut, the first and second raised regions disposed on opposite sides of the chevron.

72. A method for treating an aneurysm in a blood vessel, the method comprising:
providing a delivery catheter having a prosthesis coupled thereto, the prosthesis comprising a tubular body expandable from a contracted configuration to a radially expanded configuration, the tubular body having a total length and comprising a first section, a second section and a central section disposed therebetween, wherein each of the sections has a longitudinal length;
advancing the prosthesis toward the aneurysm;
radially expanding the prosthesis such that each of the first, the central, and the second sections expand to a diameter, wherein the central section expands to a diameter different than the expanded diameter of the first section and the expanded diameter of the second section, wherein the total length of the tubular body in the radially expanded configuration is at least 95% of the total length of the tubular body in the contracted configuration; and
removing the delivery catheter from the aneurysm.

73. The method of claim 72, wherein the step of radially expanding the prosthesis comprises:
radially expanding the first section before radially expanding the central section; and
radially expanding the central section before radially expanding the second section, wherein
the expanded diameter of the first section is greater than the expanded diameter of the central section and the expanded diameter of the central section is greater than the expanded diameter of the second section.

74. The method of claim 72, wherein the step of radially expanding the prosthesis comprises forming a stepped region between either an outer surface of the first section and an outer surface of the central section or forming a stepped region between an outer surface of the central section and an outer surface of the second section.

75. The method of claim 72, wherein the step of radially expanding the prosthesis comprises forming a substantially smooth taper from the first section to the central section and the second section.

76. The method of claim 72, wherein the step of radially expanding the prosthesis comprises radially expanding the central section after radially expanding the first section and the second section.

77. The method of claim 72, wherein the step of radially expanding the prosthesis comprises radially expanding first section and the second section before radially expanding the central section.

78. The method of claim 72, wherein the step of radially expanding the prosthesis comprises flaring at least one of the first section or the second section.

79. The prosthesis of claim 72, wherein the step of radially expanding the prosthesis comprises flaring both the first section and the second section.

80. The method of claim 72, wherein the step of radially expanding the prosthesis comprises radially expanding the second section after radially expanding the first section and the central section.

81. The method of claim 72, wherein the tubular body further comprises a fourth section disposed between the central section and the second section, and wherein the step of radially expanding the prosthesis comprises radially expanding the first section and the central section prior to radially expanding the second section and the fourth section.

82. The method of claim 72, wherein the step of radially expanding the prosthesis comprises radially expanding the central section prior to radially expanding both the first section and the second section.

83. The method of claim 72, wherein the step of radially expanding the prosthesis comprises expanding the prosthesis from a first diameter in the contracted configuration to a second diameter in the radially expanded configuration such that the ratio of the second diameter to the first diameter is greater than 1 and less than about 15.

84. The method of claim 72, wherein the step of radially expanding the prosthesis comprises expanding an expandable member disposed on the delivery catheter.

85. The method of claim 84, wherein the expandable member comprises a balloon.

86. The method of claim 72, wherein the tubular prosthesis has a diameter in the radially expanded configuration, the method further comprising maintaining at least 50% of the radially expanded diameter along at least a portion of the tubular prosthesis when an externally applied radial differential pressure of between about 60 mm Hg to about 1000 mm Hg is applied thereto.

87. The method of claim 72, further comprising forming a curve in the tubular prosthesis, the curve having a radius of 0.2 inches or more without forming a kink therein.

88. The prosthesis of claim 87, wherein the kink comprises a collapsed region of the tubular prosthesis having a diameter in the expanded configuration less than 50% of the diameter of the tubular prosthesis in the expanded configuration.

89. The prosthesis of claim 87, wherein the kink comprises a collapsed region of the tubular prosthesis having a cross-sectional area less than 50% of an uncollapsed cross-sectional area of the prosthesis.

90. The method of claim 72, wherein the prosthesis further comprises an inflatable member coupled with the tubular body, the method further comprising inflating the inflatable member.

91. The method of claim 90, wherein the inflatable member is inflated into engagement with a wall of the aneurysm.

92. The method of claim 90, wherein the inflatable member is inflated with an in situ curable polymer.

93. The method of claim 90, wherein the step of inflating comprises anchoring the inflatable member and the tubular body with the aneurysm.

94. The method of claim 90, wherein the step of inflating comprises filling the inflatable member with an in situ curable polymer to a differential pressure of 60-1000 mm Hg, and wherein the expanded prosthesis allows blood perfusion therethrough during
filling and curing of the inflatable member.

95. The method of claim 72, wherein the first section is disposed upstream of the aneurysm.

96. The method of claim 72, wherein the central section is disposed in the aneurysm.

97. The method of claim 72, wherein the second section is disposed downstream of the aneurysm.

98. The method of claim 72, wherein the delivery catheter comprises a restraining member disposed thereon and radially expanding the prosthesis comprises removing the restraining member from the tubular prosthesis.

99. The method of claim 72, wherein the aneurysm is disposed in an aorta.

100. The method of claim 72, wherein the aneurysm is disposed in an abdominal aorta.

101. The method of claim 72, wherein removing the delivery catheter comprises deflating an inflatable member disposed on the delivery catheter.

102. The method of claim 72, wherein the prosthesis comprises a therapeutic agent coupled thereto and the method further comprises delivering the therapeutic agent in a controlled manner.

103. A method of fabricating a tubular prosthesis having a longitudinal axis and axially variable characteristics, said method comprising:
fabricating a first region of the tubular prosthesis, the first region having a first set of material characteristics;
fabricating a second region of the tubular prosthesis, the second region having a second set of material characteristics; and
fabricating a third region of the tubular prosthesis, the third region having a third set of material characteristics,
wherein the first region, second region and third region are axially aligned along the longitudinal axis, and
wherein the first set of material characteristics is different than the second set of material characteristics and wherein the second set of material characteristics is different than the third set of material characteristics, and
wherein the first region radially expands before the second or the third regions when the tubular prosthesis is radially expanded.

104. The method of claim 103, wherein fabricating the first region, the second region or the third region comprises electrical discharge machining of a tube or a substantially flat sheet of material.

105. The method of claim 103, wherein fabricating the first region, the second region or the third region comprises laser cutting a tube or a flat sheet of material.

106. The method of claim 103, wherein fabricating the first region, the second region or the third region comprises photochemically etching a tube or a flat sheet of material.

107. The method of claim 103, wherein the second region is disposed between the first and third regions of the prosthesis.

108. The method of claim 103, further comprising:
fabricating a fourth region of the tubular prosthesis, the fourth region having a fourth set of material characteristics,
wherein the fourth set of material characteristics is different than the first set of material characteristics, and
wherein the fourth region radially expands after the first region of the tubular prosthesis when deployed.

109. The method of claim 103, wherein the first, second, or third set of material characteristics comprises at least one mechanical property selected from the group consisting of strut length, strut width, strut thickness, number of struts per cell, connector radius, connector thickness, connector geometry, material temper, material strength, and combinations thereof.
